(19) United States
(12) Patent Application Publication
(10) Pub. No.: US 2010/0305326 A1
(43) Pub. Date: Dec. 2, 2010

(54) Chemical Fragment Screening and Assembly Utilizing Common Chemistry for NMR Probe Introduction and Fragment Linkage

(75) Inventor: Daniel S. Sem, New Berlin, WI (US)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: Marquette University, Milwaukee, WI (US)

(21) Appl. No.: 12/792,369

(22) Filed: Jun. 2, 2010

Related U.S. Application Data
(60) Provisional application No. 61/217,616, filed on Jun. 2, 2009.

(51) Int. Cl. C07D 215/12; C07D 401/02; C07D 211/68; C07D 215/02

(52) U.S. Cl. 546/152; 546/276.4; 546/277.7; 546/285; 546/176

(57) ABSTRACT

Disclosed herein are methods related to drug development. The methods typically include steps whereby two chemical fragments are identified as binding to a target protein and subsequently the two chemical fragments are joined to create a new chemical entity that binds to the target protein.

### CROSS-REFERENCE TO RELATED APPLICATIONS

The present application claims the benefit under 35 U.S.C. §119(e) to U.S. Provisional Application No. 60/217,616, filed on Jun. 2, 2009, the contents of which are incorporated herein by reference.

### STATEMENT REGARDING U.S. GOVERNMENT SPONSORED RESEARCH OR DEVELOPMENT

This invention was made with U.S. government support under Grant No: R15 GM085739 from the National Institutes of Health. The U.S. government has certain rights in this invention.

### BACKGROUND

The field of the present invention relates to drug development. In particular, the invention relates to methods for screening and assembling chemical fragments to create new chemical entities for use as drugs.

The drug discovery process is costly and often inefficient. Combinatorial chemistry, high throughput screening and even structure-based drug design (i.e., rational drug design) methods are examples of technologies that have been introduced in the last 20 years in order to improve the efficiency of the drug discovery process. Still, the cost of drug discovery continues to rise, yet the number of new drug molecules (New Chemical Entities, or NCEs) introduced onto the market is not increasing in parallel. In fact, the pipeline of new drugs coming from the pharmaceutical industry is shrinking.

Another drug discovery technology, introduced in the early 1990s as a way to improve the efficiency of the drug discovery process, is termed “fragment based” drug design, whereby two smaller chemical fragments (<400 g/mol and more preferably <350 g/mol) are identified that bind close to each other on the surface of a target protein for therapy. This approach, termed SAR by NMR, was pioneered at Abbott Laboratories. Once it is established that these two fragments, namely fragment A and fragment B, bind close to each other on the target protein, the fragments are then chemically joined or tethered. There are advantages to this approach whereby the newly created chemical entity (A-B) has a higher affinity for the target protein than either fragment A or fragment B and many successes have been reported. However, one significant limitation to this fragment-based approach is that even though it may be known that two fragments (A and B) should be linked to form a new chemical entity (A-B), it is often chemically difficult or impossible to link them. As such, better methods for identifying and chemically combining fragments are needed in order to provide new chemical entities.

### SUMMARY

Disclosed herein are methods related to drug development. The methods typically include steps whereby two chemical fragments are identified as binding to a target protein and subsequently the two chemical fragments are joined to create a new chemical entity that binds to the target protein.

In some embodiments, the disclosed methods are utilized to create a chemical compound, namely A-B, from two chemical fragments, namely A and B, where the chemical compound binds to a target protein. The methods may include the following steps: (a) methylating one of the chemical fragments, namely A, at one or more positions to obtain a ¹³CH₃-methylated analog of A, namely A-¹³CH₃, by performing an alkylation reaction; (b) forming a mixture comprising: (1) A-¹³CH₃; (2) the other chemical fragment, namely chemical fragment B, which comprises a methyl group (e.g., an allylic or a benzylic methyl group), and (3) the target protein; (c) determining whether both A-¹³CH₃ and B bind to the target protein in the mixture such that the methyl group of A-¹³CH₃ and the methyl group of B are located no more than 5 angstroms apart; and if so (d) performing the alkylation reaction of step (a) using A and B as reagents in order to covalently attach A and B via the methyl group carbon atom of B to obtain the chemical compound A-B. Typically, fragment A and fragment B are chosen for the method such that the chemical reaction that ultimately will be used to join fragment A and fragment B can be easily performed, typically via a nucleophilic displacement reaction, such as an S_{N}2 reaction.

In order to determine whether both A-¹³CH₃ and B bind to the target protein in the mixture such that the methyl group of A-¹³CH₃ and the methyl group of B are located no more than 5 angstroms apart, nuclear magnetic resonance (NMR) may be performed on the mixture in order to determine whether a Nuclear Overhauser Effect (NOE) is occurring. In some embodiments, determining whether an NOE is occurring may include performing a ¹³C-filtered measurement either in a single dimension or in two dimensions.

The mixture utilized in the methods includes: (1) A-¹³CH₃; (2) the chemical fragment B, which comprises a methyl group (e.g., an allylic or benzylic methyl group), and (3) the target protein. In some embodiments, the mixture comprises at least 10 times more of A-¹³CH₃ and at least 10 times more of the chemical fragment B than the target protein on a molar basis. These conditions are permissible for what is referred to in the art as a transferred NOE study.

The mixture includes a target protein, for example, the mixture may include a biological sample that includes the target protein and optionally includes a non-target protein. Suitable biological samples may include extracts of human tissue (e.g., extracts of brain tissue, heart tissue, or liver tissue). Extracts may be enriched for one or more target proteins by purification methods that include affinity chromatography using a column that comprises a known ligand for the target protein. Suitable target proteins, for example, may include a KCNQ (Kv7) channel protein. A suitable method for purifying KCNQ (Kv7) may include passing a brain tissue extract over an affinity column comprising a covalently attached drug or ligand known to bind to KCNQ (Kv7) in a chromatographic purification method. Then, the column may be washed to remove non-binding proteins. The bound proteins then may be eluted, including KCNQ (Kv7) protein, using a solution containing the drug or ligand as an eluent. In some embodiments of the methods, the methods further include performing NMR on a mixture formed from: (1) A-¹³CH₃; (2) the other chemical fragment, B, which comprises a methyl group, and (3) the biological sample after the target protein has been removed from the biological sample. The NMR results from the mixture that includes the target protein may be compared to the NMR results from the mixture that does not include the target protein as a control. In particular, NMR measurements may be compared from the eluate and the wash steps in the chromatographic purification method of KCNQ or another target protein as described above.

In some embodiments of the methods, the chemical fragment A is methylated at a carbon atom to create an alkyl bond, an oxygen atom to create an ether bond, or at a sulfur atom to create a thioether bond. In further embodiments, the chemical fragment B comprises an allylic methyl group or a benzylic methyl group. For example, in step (a) of the disclosed methods, the chemical fragment A may be methylated at a carbon, oxygen, or sulfur atom. Further, in step (d) the chemical fragment A may be covalently attached to chemical fragment B via forming a bond between the carbon, oxygen, or sulfur atom of chemical fragment A and the methyl group carbon atom of chemical fragment B thereby forming a C—C bond, an O—C bond, or a S—C bond, respectively.

Suitable compounds for use as the chemical fragment A may include, but are not limited to compounds capable of forming carbanions, e.g., where a carbon atom of the chemical fragment. A is deprotonated and the resulting carbanion subsequently is methylated. Suitable compounds for use as the chemical fragment A may include, but are not limited to compounds comprising alcohol groups, e.g., where the oxygen atom of the alcohol group is deprotonated and the resulting oxygen anion subsequently is methylated to form an ether. Suitable compounds for use as the chemical fragment A may include, but are not limited to compounds comprising thiol groups, e.g., where the sulfur atom of the thiol group is deprotonated and the resulting sulfur anion subsequently is methylated to form a thioether.

In some embodiments, the chemical fragment A has a formula selected from:

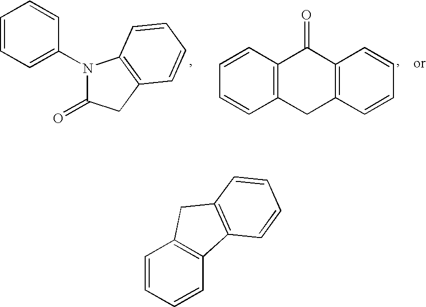

The chemical fragment A is methylated at one or more positions and may be di-methylated. In some embodiments, a di-methylated chemical fragment A has a formula selected from:

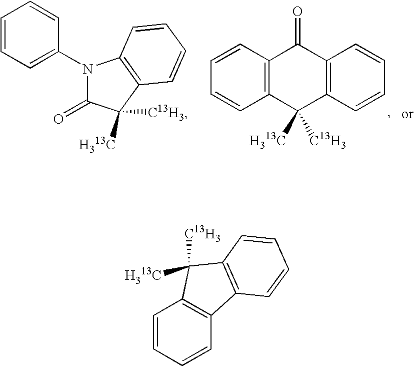

Suitable compounds for use as the chemical fragment B typically include a pendant methyl group. Suitable compounds for use as the chemical fragment B, may include, but are not limited to compounds selected from list of compound in Tables 2 and 3. In some embodiments, the chemical fragment B is a methyl substituted pyridine compound. In further embodiments, the chemical fragment B includes a fused ring moiety selected from a quinoline, an isoquinoline, and an acridine. In even further embodiments, the chemical fragment B has a formula selected from:

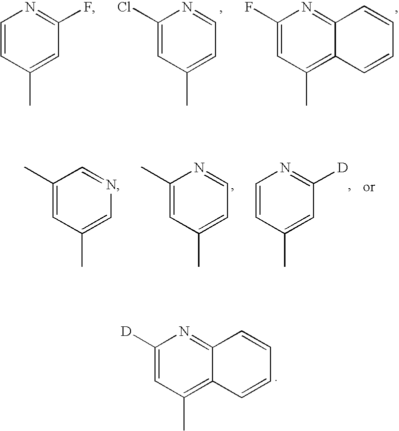

The disclosed methods typically utilize an alkylation reaction for methylating the chemical fragment. A. Suitable alkylation reactions may include a step whereby nucleophilic substitution on an alkyl halide occurs. In some embodiments, the alkylation reaction may comprise the following steps: (i) reacting the chemical fragment A with a base (e.g., a strong base such as NaH, or NaNH₂ or a weaker base such as NaOH) under conditions whereby the chemical fragment A is deprotonated at a nucleophilic atom; and (ii) reacting the deprotonated chemical fragment A with a methyl halide thereby methylating the chemical fragment A at the nucleophilic atom. The methyl halide may include a ¹³C. The alkylation reaction may include (i) reacting the chemical fragment A with a base (e.g., a strong base such as NaH, or NaNH₂ or a weaker base such as NaOH) under conditions whereby the chemical fragment A is deprotonated at a carbon atom (i.e., removing one or more hydrogen atoms to create a carbanion), an alcohol (i.e., to create an oxygen anion), or a thiol (i.e., to create a sulfur anion); and (ii) reacting the deprotonated chemical fragment A with a methyl halide thereby methylating the chemical fragment A at the nucleophilic atom. Suitable solvents for such a methylation reaction may include DMF, DMSO, and other polar aprotoic solvents. The methylated chemical fragment A subsequently may be utilized in the NMR methods contemplated herein.

The disclosed methods typically utilize a common alkylation reaction for covalently attaching the chemical fragment A and the chemical fragment B via the methyl group carbon atom of B in order to obtain a chemical compound A-B. In some embodiment the alkylation reaction for covalently attaching the chemical fragment A and the chemical fragment B includes the following steps: (i) reacting the chemical fragment A with a base (e.g., a strong base such as NaH, or NaNH₂ or a weaker base such as NaOH) under conditions whereby the chemical fragment A is deprotonated at a nucleophilic atom (e.g., at a nucleophilic carbon such as an allylic or benzylic carbon; at a nucleophilic oxygen of an alcohol group; or at a nucleophilic sulfur atom of a thiol group); (ii) halogenating the methyl group of the chemical fragment B to obtain a derivative of chemical fragment B having a halogenated methyl group; and (iii) reacting the deprotonated chemical fragment A with the derivative of chemical fragment B having the halogenated methyl group, thereby forming a bond between the deprotonated nucleophilic atom of the chemical fragment A and the methyl group carbon of the chemical fragment B (e.g., a —C—C— bond, a —O—C— bond, or a —S—C— bond). In some embodiments, halogenation of the methyl group of the chemical fragment B may be performed by methods that include, but are not limited to, reacting the chemical fragment B with N-bromosuccinimide (NBS) or N-chlorosuccinimide (NCS).

In further embodiments, the disclosed methods may be practiced in order to create a chemical compound, namely A-B, from two chemical fragments, namely A and B, where the chemical compound binds to a KCNQ (Kv7) channel protein. The method may include the following steps: (a) methylating one of the chemical fragments, A, at one or two positions (which may be controlled using stoichiometry of reactants) to obtain a ¹³CH₃-methylated analog of A, namely A-¹³CH₃, by performing an alkylation reaction, where a di-methylated derivative of chemical fragment A has a formula selected from:

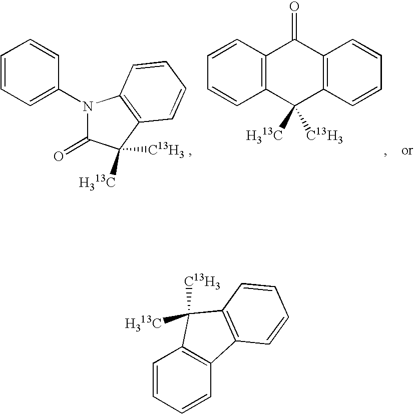

(b) forming a mixture comprising: (1) A-¹³CH₃; (2) the other chemical fragment, B, which may be selected from compounds listed in Tables 2 or 3, and (3) the KCNQ (Kv7) channel protein; (c) determining whether both A-¹³CH₃ and B bind to the target protein in the mixture such that the methyl group of A-¹³CH₃ and the methyl group of B are located no more than 5 angstroms apart; and if so (d) performing the alkylation reaction of step (a) using A and B as reagents in order to covalently attach A and B via the nucleophilic atom of A (after deprotonation) and the methyl group carbon atom of B (after halogenation) to obtain the chemical compound A-B.

### BRIEF DESCRIPTION OF THE DRAWINGS

FIG. 1. NMR-based fragment assembly of the prior art utilizing a protein kinase as a target protein. A. Structure of a protein kinase showing the drug lead SB203580 bound in the active site, and the adjacent binding pocket where peptide binds. The peptide occupies part of the so-called specificity pocket, which is variable between related kinase isoforms. B. Closeup view of the specificity pocket's location proximate to the SB203580 ligand, such that if another ligand fragment occupied that site, it could be chemically linked to SB203580, to provide more affinity and specificity to the protein kinase drug target protein shown. C. Chemical structure of a modified form of SB203580, showing how NMR experiments (NOE measurements) can detect fragments that bind within 5 angstroms of each other.

FIG. 2. Illustrative methods for synthesizing NMR probes for fragment screening in order to identify groups to covalently attach to the validated scaffold.

FIG. 3. NOE-based screening (¹³C-filtered ¹H-¹H NOEs) to identify interacting fragments that bind to the KCNQ channel protein from brain, a strategy that may be utilized to prepare derivatives of DMP543 where the screening utilizes a fragment of DMP543 and derivatives thereof.

FIG. 4. Illustration of fragment assembly successes, using SAR by NMR, from Abbott laboratories, which led to drugs that have entered human clinical trials. (See Hajduk and Greer, Nature Reviews—Drug Discovery, Vol. 6, March 2007, 211-219).

FIG. 5. The three drugs from which the A fragments in FIGS. 2 and 3 were derived.

FIG. 6. Two additional drugs from the top 200 selling drugs, which were synthesized in a manner involving an intermediate that possessed a nucleophilic O, S, or C atom.

FIG. 7. Methylation of glitazone at a nucleophilic oxygen atom.

### DETAILED DESCRIPTION

Disclosed herein are methods related to drug development. The methods typically include steps whereby two chemical fragments are identified as binding to a target protein and subsequently, the two chemical fragments are joined to create a new chemical entity that binds to the target protein.

The methods may be described using several definitions as discussed below.

Unless otherwise specified or indicated by context, the terms “a”, “an”, and “the” mean “one or more.” In addition, singular nouns such as “chemical fragment” and “target protein” should be interpreted to mean “one or more chemical fragments” and “one or more target proteins,” unless otherwise specified or indicated by context.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≦10% of the particular term and “substantially” and “significantly” will mean plus or minus >10% of the particular term.

As used herein, the terms “include” and “including” have the same meaning as the terms “comprise” and “comprising.”

As disclosed herein, methods are utilized to create a chemical compound, namely A-B, from two chemical fragments, namely A and B, where the chemical compound binds to a target protein. The methods may include the following steps: (a) methylating one of the chemical fragments, namely A (which otherwise may be referred to herein as a “scaffold molecule” or a “core molecule”), at one or more positions to obtain a ¹³CH₃-methylated analog of A, namely A-¹³CH₃, by performing an alkylation reaction; (b) forming a mixture comprising: (1) A-¹³CH₃; (2) the other chemical fragment, namely chemical fragment B, which comprises an allylic or benzylic methyl group (and otherwise may be referred to herein as a “pendant group molecule”), and (3) the target protein (e.g., where the mixture comprises a biological sample comprising the target protein and optionally a non-target protein); (c) determining whether both A-¹³CH₃ and B bind to the target protein in the mixture such that the methyl group of A-¹³CH₃ and the methyl group of B are located no more than 5 angstroms apart; and if so (d) performing the alkylation reaction of step (a) using A as a reagent (optionally after A has been deprotonated) and B as a reagent (after B has been halogenated) in order to covalently attached A and B via the methyl group carbon atom of B to obtain the chemical compound A-B.

A “biological sample” as used herein means any solid or liquid material that includes a target protein. A biological sample may include material obtained from an animal (e.g., human) or a non-animal source (e.g., bacteria, mycobacteria, and fungi). A biological sample may include a human biological sample, which may include but is not limited to, neurological tissue (e.g., brain), liver tissue, heart tissue, breast tissue, kidney tissue, lung tissue, and muscle tissue. A biological sample may include human body fluids (e.g., blood or blood products). A biological sample also may have been subjected to partial purification using chromatographic methods, such as affinity chromatography where a chromatographic resin that comprises a known ligand for the target protein is used.

A “target protein” as used herein is a protein to which an existing drug or chemical compound binds, thereby modulating biological activity of the protein and causing a therapeutic effect. A “non-target protein” or an “anti-target protein” is a protein to which an existing drug or chemical compound binds, thereby modulating biological activity of the protein and causing a side effect. For example, target proteins useful for the methods disclosed herein may include target proteins that are therapeutic targets for treating psychiatric disorders. Suitable target proteins include the proteins that form the KCNQ (Kv7) channel in neural tissue of human. The “KCNQ channels” alternatively referred to as the “Kv7 channels” are a small family of voltage-gated potassium channel subunits that are encoded by the KCNQ genes (KCNQ1-5). (See, e.g., Robbins, J. (2001). Pharmacol. Ther. 90, 1-19; and Jentsch T. J. (2000) Nat. Rev. Neurosci. 1, 21-30, the contents of which are incorporated by reference in their entireties). Modulation of KCNQ channel activity has been suggested to have therapeutic potential. (See, e.g., Wulff et al., Nature Reviews, Drug Discovery, Volume 8, Pages 982-1001, December 2009; Brown, J. Physiol. 586.7 (2008) pp 1781-1783; Gribkoff, Expert Opin. Ther. Targets (2008) 12 (5):565-581; Xiong et al., Trends in Pharmacological Sciences, 2007, 29 (2), pages 99-107; and Gribkoff, Expert Opin. Ther. Targets (2003) 7 (6):737-748; the content of which is incorporated herein by reference in their entireties).

The present methods utilize chemical fragments which subsequently are assembled to create new chemical compounds (i.e., new chemical entities (NCEs)). As used herein, a “chemical fragment” is a chemical compound intended to be covalently attached to a second chemical fragment. Exemplary chemical compounds for use as chemical fragments in the disclosed methods include those listed in Tables 1-3.

Chemical fragments for use in the disclosed methods may be obtained based on reviewing existing drugs and chemical compounds and identifying common moieties in the existing drugs and chemical compounds. The identified common moieties may be utilized as a chemical fragment in the present methods and combined with another chemical fragment to obtain a new chemical compound provided that the chemical fragments have or can be modified to have the properties of chemical fragment A and chemical fragment B as described herein. Existing drugs and chemical compounds that may be utilized in the methods disclosed herein include those drugs available from commercial libraries such as The Prestwick Chemical Library® collection (Prestwick Chemical, Inc.) (See Table 4.) Other existing drugs and chemical compounds that may be utilized in the methods disclosed herein include those drugs available from The Spectrum Collection (Microsource Discovery System, Inc.). (See Table 5. See also J. Virology 77:10288 (2003) and Ann. Rev. Med. 56:321 (2005), the contents of which are incorporated herein by reference in their entireties). Other existing drugs and chemical compound that may be utilized in the method disclosed herein include those drugs available from the Sequoia collection at its website or those drugs published by Advanstart Medical Economics: Top 200 Drugs, A 5-Year Compilation (2009), the contents of which are incorporated by reference herein in their entireties. (See Table 6). Other sources of chemical fragments include the fragment-like subset of the ZINC database (Irwin and Shoichet (2005), J. Chem. Inform. Model. 45, 177-182, the content of which is incorporated herein by reference in its entirety).

The disclosed methods typically utilize at least two fragments, namely, fragment A and fragment B. Typically, the fragments have a molecular weight that is less than about 400 g/mol and preferably less than about 350 g/mol. Further, fragments preferably have ≦3 hydrogen-bond donors, ≦3 hydrogen-bond acceptors, and do not contain chemical groups known to serve as poor drug leads, such as Michael acceptors and highly electrophilic groups.

Fragment A typically comprises a nucleophilic atom. Suitable nucleophiles include carbon atoms that form a carbon nucleophiles (i.e., carbanions), oxygen atoms (e.g., which are part of an alcohol group), and sulfur atoms (e.g., which are part of a thiol group). The nucleophile is capable of being methylated, for example by reacting with a compound having a halogenated alkyl group (preferably a primary carbon in order to facilitate an S_{N}2 reaction) under basic reaction conditions whereby the carbanion nucleophile forms. Where the carbon nucleophile (i.e., carbanion) is formed under basic conditions (e.g., with sodium amide or NaH) and reacted with ¹³CH₃X, where X is a halide, suitable solvents may include, but are not limited to DMF, DMSO, and other polar, aprotoic solvents.

Suitable nucleophiles may include carbon nucleophiles such as carbon atoms adjacent to (alpha to) one or two carbonyl (C═O) groups, which makes the C—H proton on that alpha carbon more acidic due to tautomerization reactions. A C—H group adjacent to a carbon-carbon double bond, such as in a benzene ring and an allylic compound, are also more acidic, such that a carbon nucleophile (carbanion) can form. Carbon nucleophiles well known in the art include malonate esters, which are used as synthetic precursors. Often, drugs are synthesized using an intermediate chemical structure that contains a carbon nucleophile, and in this case the intermediate that contains the carbon nucleophile can be methylated to make a fragment A-¹³CH₃NMR probe for use in the present methods. The carbanion nucleophile of chemical fragment A may be covalently attached to chemical fragment B as follows. In step (d) of the presently disclosed methods, the chemical fragment A may be covalently attached to chemical fragment B via forming a bond between the carbon nucleophile of chemical fragment A and the methyl group carbon atom of chemical fragment B (thereby forming an C—C bond between chemical fragment A and chemical fragment B). For example, a chemical reaction may be readily achieved where chemical fragment B comprises an allylic or benzylic methyl group, which can be readily chlorinated, brominated, or iodinated (e.g., by reacting chemical fragment B with N-chloro-succinamide, N-bromo-succinamide, or N-iodo-succinamide, respectively) to form a halogenated chemical fragment B having a halogenated, allylic or benzylic methyl group (i.e., CH₂—X where X=Br, Cl or I). The halogenated chemical fragment B may then be reacted with a chemical fragment A via a nucleophilic substitution at the carbon nucleophile of chemical fragment A.

Other suitable nucleophiles include nucleophilic oxygen atoms (e.g., as part of an alcohol group) or a nucleophilic sulfur atoms (e.g., as part of a sulfur group). Suitable thiol compounds for use in the present methods include thiol compounds listed in the database maintained by the Chemical Proteomics Facility of Marquette University (accessed on Jun. 1, 2010), a partial list of which is provided in Table 1.

In some embodiments of the disclosed methods, in step (a) of the disclosed methods, the chemical fragment A may be methylated on the alcohol or thiol group in order to form an ether or a thioether compound, respectively. Further, in step (d) the chemical fragment A may be covalently attached to chemical fragment B via forming a bond between the oxygen atom or sulfur atom of chemical fragment A and the methyl group carbon atom of chemical fragment B (thereby forming an O—C bond or a S—C respectively between chemical fragment A and chemical fragment B). For example, a chemical reaction may be readily achieved where chemical fragment B comprises an allylic or benzylic methyl group, which can be readily chlorinated, brominated, or iodinated (e.g., by reacting chemical fragment B with N-chloro-succinamide, N-bromo-succinamide, or N-iodo-succinamide, respectively) to form a halogenated chemical fragment B having a halogenated, allylic or benzylic methyl group (i.e., CH₂—X where X=Br, Cl or I). The halogenated chemical fragment B may then be reacted with a chemical fragment A having an —OH or —SH group via a nucleophilic substitution reaction, which produces the desired fusion of the two fragments having a —C—O—C— linkage (ether linkage) or a —C—S—C— linkage (thioether linkage). Suitable compounds for fragment A may include any compound that has an alcohol or thiol group that can then be methylated to form an ether or a thioether.

In some embodiments, a suitable fragment A having a nucleophilic oxygen atom or nucleophilic sulfur atom may be prepared by first halogenating a compound having an allylic or benzylic methyl group at the methyl group. Subsequently, the halogenated compound is reacted with an oxy anion (e.g., NaOH) or a thiol anion (e.g., NaSH) which replaces the halogen in a nucleophilic substitution reaction. The compounds in Tables 2 and 3 having allylic or benzylic methyl groups may be reacted accordingly to obtain a chemical fragment A having a nucleophilic oxygen atom or nucleophilic sulfur atom.

Fragments that are suitable for the use in the present methods (or a library of fragments) may be selected by criteria that include the “Rule of 3.” (See, e.g., Lipinski, C. A. Drug Discovery Today: Technologies 2004, 1, 337-341; and Erlanson, D. A.; Braisted, A. C.; Raphael, D. R.; Randal, M.; Stroud, R. M.; Gordon, E. M.; Wells, J. A. Proc. Natl. Acad. Sci. U.S.A. 2000, 97, 9367-9372; the contents of which are incorporated by reference in their entireties). Fragment libraries, as contemplated herein, preferably are diverse. One method of assessing diversity of the library is to compare it to another library, using principal component-based measures of diversity. (See, e.g., Fink, T.; Reymond, J. L. J. Chem. Inf. Comput. Sci. 2007, 47, 342-353; the content of which is incorporated by reference herein in its entirety). Fragments for use in the present methods preferably are soluble. (See, e.g., Olah, M. M.; Bologa, C. G.; Oprea, T. I. Current Drug Discovery Technologies 2004, I, 211-220; Siegal, G.; AB, E.; Schultz, J. Drug Discov. Today 2007, 12, 1032-1039; and Lepre, C. A. Drug Discov. Today 2001, 6, 133-140; the contents of which are incorporated by reference in their entireties). Solubility can be measured or estimated in many ways. (See, e.g., 20. Lipinski, C. A.; Lombardo, F.; Dominy, B. W.; Feeney, P. J. Advanced Drug Delivery Revies 2001, 46, 3-26; the content of which is incorporated by reference in its entirety). In some embodiments, fragments for the presently disclosed methods may be selected to include no atoms other than C, O, H, N, S, P, F, Cl, Br, or I. In further embodiments, fragments for the presently disclosed methods may be selected to include no functional groups that are reactive with proteins. For example, fragments may be selected to include none of the following functional groups: Michael acceptors, anhydrides, epoxides, alkyl halides, acyl halides, imines, aldehydes, or aliphatic ketones. Some compounds meeting this criteria are listed in a database maintained by the Chemical Proteomics Facility of Marquette University at its website (accessed on Jun. 1, 2010), a partial list of which is provided in Table 1.

Suitable existing drugs or chemical compounds for the methods contemplated herein may modulate KCNQ (Kv7) channel activity. These include compounds that bind to the KCNQ (Kv7) channel and inhibit or alternatively activate or enhance KCNQ (Kv7) channel activity. Suitable compounds may inhibit KCNQ (Kv7) channel activity by blocking, closing, or otherwise inhibiting a KCNQ (Kv7) channel from facilitating passage of ions from one side of a membrane to the other side of the membrane in which the KCNQ (Kv7) channel is present. KCNQ (Kv7) channel activity and modulation thereof, including inhibition thereof, may be assessed by methods described in the art (e.g., patch clamp analysis, see, e.g., Bal et al., J. Biol. Chem. 2008 283 (45):30668-30676; Wu et al., J. Neurophysiol. 2008 100 (4):1897-1908; Kasten et al., J. Physiol. 2007 584 (Pt. 2):565-582; Jia et al, J. Gen. Physiol. 2006 131 (6):575-587; and Wladyka et al., J. Physiol. 2006 575 (Pt. 1):175-189; the contents of which are incorporated by reference in their entireties).

Compounds that modulate KCNQ (Kv7) channel activity are known in the art and may include KCNQ (Kv7) channel activity inhibitors or alternatively KCNQ (Kv7) channel activity activators. KCNQ (Kv7) channel activity inhibitors may include but are not limited to linopirdine (Dupont), XE991 (Dupont), DMP543 (Dupont), d-tubocurarine, verapamil, 4-aminopurine, CP-339818 (Pfizer), UK-78282 (Pfizer), correolide (Merck), PAP-1 (UC-Davis), clofazimine, Icagen (Eli Lilly), AVE-0118 (Sanofi-Aventis), Vernakalant (Cardiome), ISQ-1 (Merck), TAEA (Merck), DPO-1 (Merck), azimilide (Proctor and Gamble), MHR-1556 (Sanofi-Aventis), L-768673 (Merck), astemizole, imipramine, dofetilide, NS1643 (Neurosearch), NS3623 (Neurosearch), RPR26024 (Sanofi-Aventis), PD307243 (GlaxoSmithKline), and A935142 (Abbott Laboratories). KCNQ (Kv7) channel activity activators may include but are not limited to retigabine, flupirtine, ICA-27243 (Icagen), ICA-105665 (Icagen), diclofenac, NH6, niflumic acid, mefenamic acid, and L364373 (Merck). These compounds and other compounds that modulate KCNQ (Kv7) channel activity are disclosed in Wulff et al., Nature Reviews, Drug Discovery, Volume 8, Pages 982-1001, December 2009 (the content of which is incorporated herein by reference in its entirety).

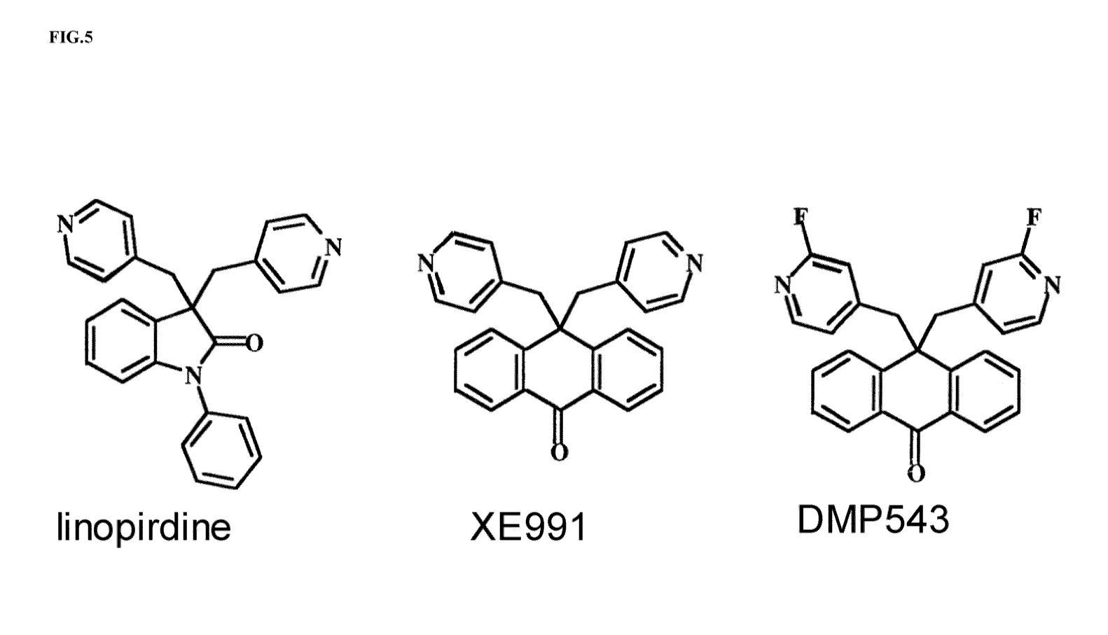

A suitable drug or compound for the methods contemplated herein may include DMP543 or analogs or derivatives thereof (e.g., analogs or derivatives thereof that inhibit KCNQ (Kv7) channel activity). Referring to the PubChem Database provided by the National Center for Biotechnology Information (NCBI) of the National Institute of Health (NIH), DMP543 is referenced by compound identification (CID) number 9887884 (which entry is incorporated herein by reference in its entirety). (See also FIG. 5.) Analogs or derivative of DMP543 may include salts, esters, amides, or solvates thereof. Furthermore, analogs or derivatives of DMP543 may include “similar compounds” or “conformer compounds” as defined at the PubChem Database, which include but are not limited to compounds referenced by CID Nos.: 9801773, 10644338, 9930525, 19606104, 10926895, 10093074, 10093073, 45194349, 19606090, 19606069, 19606087, 19606071, 19606104, 19606084, 19606108, 19606110, 19606109, and 15296110, which entries are incorporated herein by reference in their entireties.

A suitable drug or compound for the methods contemplated herein may include XE991 or analogs or derivatives thereof (e.g., analogs or derivatives thereof that inhibit KCNQ (Kv7) channel activity). Referring to the PubChem Database provided by the National Center for Biotechnology Information (NCBI) of the National Institute of Health (NIH), XE991 is referenced by compound identification (CID) number 656732 (which entry is incorporated herein by reference in its entirety). (See also FIG. 5.) Analogs or derivative of XE991 may include salts, esters, amides, or solvates thereof. Furthermore, analogs or derivatives of XE991 may include “similar compounds” or “conformer compounds” as defined at the PubChem Database, which include but are not limited to compounds referenced by CID Nos.: 45073462, 17847140, 11122015, 19922429, 19922428, 15678637, 328741, 45234820, 45053849, 45053848, 42194630, 42194628, 21537929, 19922433, 14941569, 15678632, and 409154, which entries are incorporated herein by reference in their entireties.

The present methods may be practiced in order to identify derivatives or analogs of DMP543 or XE 991 where, in the methods, the chemical fragment A has a formula:

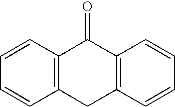

and a di-methylated derivative of A-¹³CH₃ has a formula:

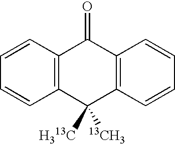

A suitable compound for the methods contemplated herein may include linopirdine or analogs or derivatives thereof (e.g., analogs or derivatives thereof that inhibit KCNQ (Kv7) channel activity). Referring to the PubChem Database provided by the National Center for Biotechnology Information (NCBI) of the National Institute of Health (NIH), linopirdine is referenced by compound identification (CID) number 3932 (which entry is incorporated herein by reference in its entirety). (See also FIG. 5.) Analogs or derivative of linopirdine may include salts, esters, amides, or solvates thereof. Furthermore, analogs or derivatives of linopirdine may include “similar compounds” or “conformer compounds” as defined at the PubChem Database, which include but are not limited to compounds referenced by CID Nos.: 11015296, 10993167, 454643, 454641, 45114239, 23581818, 14209557, 14209555, 14209553, 10549571, 9832106, 14209556, 10764944, 454654, 19438999, 14960217, 14209554, 11823673, 14209559, 15284399, 19438967, 19438958, 19438948, 19438961, 9865313, 19104987, 15296097, 19438997, 15346939, 11823673, 15284397, 15296101, 15284414, and 10476777, which entries are incorporated herein by reference in their entireties.

The present methods may be practiced in order to identify derivatives or analogs of linopirdine where, in the methods, the chemical fragment A has a formula:

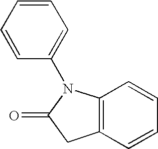

and a di-methylated derivative of A-¹³CH₃ has a formula:

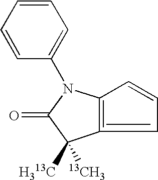

Suitable compounds for use as the chemical fragment B typically include a pendant methyl group. Suitable compounds for use as the chemical fragment B, may include, but are not limited to compounds selected from list of compound in Tables 2 and 3. In some embodiments, the chemical fragment B includes an allylic carbon, a benzylic carbon, or a pyridinyl carbon. For example, a suitable chemical fragment B may be a methyl substituted pyridine compound. The chemical fragment B may includes a single carbocyclic ring or a single heterocyclic ring, which single ring is substituted at one or more carbon atoms with a methyl group. Alternatively, the chemical fragment B may include fused carbocylic rings, heterocyclic rings, or combinations thereof, which fused rings are substituted at one or more positions with a methyl group. Suitable multiple fused ring moieties that may be present in the chemical fragment B include, but are not limited to a quinoline, an isoquinoline, and an acridine. The chemical fragment B includes at least one pendant methyl group and further may be substituted at one or more positions with halogen (F, Cl, Br, or I). In even further embodiments, the chemical fragment B has a formula selected from:

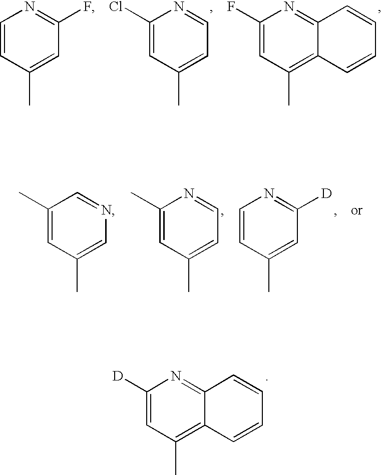

In the present methods, in order to determine whether both A-¹³CH₃ and B bind to the target protein in the mixture such that the methyl group of A-¹³CH₃ and the methyl group of B are located no more than 5 angstroms apart, a nuclear magnetic resonance (NMR) experiment may be performed on the mixture in order to determine whether a Nuclear Overhauser Effect (NOE) is occurring. An NOE is an NMR signal that represents transfer of magnetization, often between two proton atoms, and can only occur if the two atoms are within 5 angstroms of each other. The NOE that is measured is typically of two types, referred to as either steady state or transient. NMR experiments showing NOEs can typically be gathered in 2-dimensional or in 1-dimensional spectral format, and sometimes in 3-dimensional format. In some embodiments, determining whether an NOE is occurring may include performing a ¹³C-filtered measurement either in a single dimension or in two dimensions, whereby the NOE that is observed is only between: (a) the proton that is directly bonded to the ¹³C atom, and (b) any other proton, as long is it is within 5 angstroms of the ¹³C-attached proton.

NMR-based fragment assembly has been utilized in the prior art to prepare new chemical compounds. (See Hajduk and Greer (2007), “A decade of fragment-based drug design: strategic advances and lessons learned.” Nature Reviews Drug Disc. 6, 211-219; the content of which is incorporated by reference herein in its entirety). NOEs observed between fragments of an existing drug lead (SB203580) and new fragments in the presence of p38α MAP kinase indicated that these fragments bound to p38α MAP kinase and suggested a new compound to make via covalently attaching this fragments. (See Sem D S (2006) Fragment-based Approaches in Drug Discovery (Jahnke and Erlanson, Ed.), pp 163-196; the content of which is incorporated herein by reference in its entirety). These new compounds were suggested as being useful for treating rheumatoid arthritis where the new compound bound to p38α MAP kinase with a K_{d} of less than 10 nM (Sem, 2006; and U.S. Pat. No. 7,653,490; the contents of which are incorporated herein by reference in their entireties). This present methods improve fragment-based drug design of the prior art by using the same chemistry (same type of chemical reaction) to join the two fragments (A and B) that was used to introduce the NMR probe (e.g. ¹³C labeled method group) into one of the fragments. Accordingly, chemical linkage of fragments A and B will no longer be a bottleneck in fragment-based drug discovery as in current methods.

### Illustrative Embodiments

The following embodiments are illustrative and not intended to limit the claimed subject matter.

### Embodiment 1

A method for creating a chemical compound, namely A-B, from two chemical fragments, namely A and B, wherein the chemical compound binds to a target protein, the method comprising: (a) methylating one of the chemical fragments, A, at one or more positions (e.g., at nucleophilic atoms) to obtain a ¹³CH₃-methylated analog of A, namely A-¹³CH₃, by performing an alkylation reaction; (b) forming a mixture comprising: (1) A-¹³CH₃; (2) the other chemical fragment, B, which comprises an allylic or benzylic methyl group, and (3) the target protein; (c) determining whether both A-¹³CH₃ and B bind to the target protein in the mixture such that the methyl group of A-¹³CH₃ and the methyl group of B are located no more than 5 angstroms apart; and if so (d) performing the alkylation reaction of step (a) using A and B as reagents in order to covalently join A and B via the methyl group carbon atom of B to obtain the chemical compound A-B, optionally where the methyl of B has been halogenated with Cl, Br, or I and the nucleophilic atom of A attacks the carbon of the allylic or benzylic methyl group of B, displacing the halogen in a substitution reaction.

### Embodiment 2

The method of embodiment 1, wherein step (c) comprises performing nuclear magnetic resonance on the mixture and determining whether a Nuclear Overhauser Effect (NOE) is occurring (e.g., between protons on fragment A and protons on fragment B).

### Embodiment 3

The method of embodiment 2, wherein determining whether an NOE is occurring comprises performing a ¹³C-filtered measurement either in a single dimension or in two dimensions and optionally determining that the NOE involves the proton that is directly bonded to the ¹³C atom.

### Embodiment 4

The method of any of embodiments 1-3, wherein the mixture further comprises a biological sample that comprises the target protein.

### Embodiment 5

The method of embodiment 4, further comprising performing nuclear magnetic resonance on a mixture formed from: (1) A-¹³CH₃; (2) the other chemical fragment, B, which comprises a methyl group, and (3) the biological sample after the target protein has been removed from the biological sample.

### Embodiment 6

The method of embodiment 4, wherein the biological sample comprises an extract of brain tissue, heart tissue, kidney tissue, or liver tissue.

### Embodiment 7

The method of any of embodiments 1-6, wherein the target protein is a KCNQ (Kv7) channel protein.

### Embodiment 8

The method of any of embodiments 1-7, wherein the chemical fragment A comprises a nucleophilic atom selected from a nucleophilic carbon (e.g., an allylic carbon or a benzylic carbon), a nucleophilic oxygen (e.g., —OH), or a nucleophilic sulfur (e.g., —SH) and the chemical fragment A is methylated at the nucleophilic atom in step (a) and the chemical fragment A is covalently attached to chemical fragment B via forming a bond between the nucleophilic atom of chemical fragment A and the methyl group carbon atom of chemical fragment B in step (d) (e.g., after the methyl group of chemical fragment B has been halogenated).

### Embodiment 9

The method of any of embodiments 1-8, wherein the chemical fragment A is a compound selected from the list of compounds in Table 1.

### Embodiment 10

The method of any of embodiments 1-9, wherein the chemical fragment A has a formula selected from:

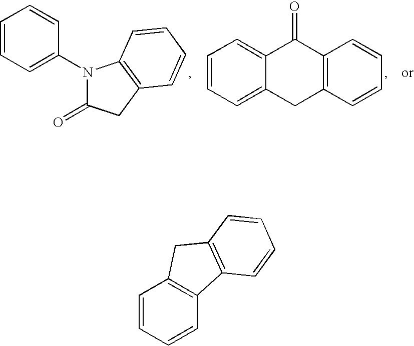

### Embodiment 11

The method of any of embodiments 10, wherein chemical fragment A is methylated at one or more positions, and the di-methylated chemical fragment A has a formula selected from:

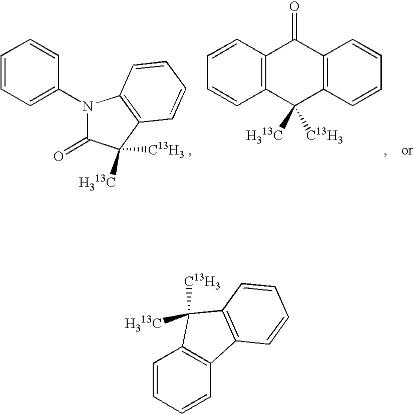

### Embodiment 12

The method of any of embodiments 1-9, wherein the chemical fragment B is a compound selected from list of compound in Tables 2 and 3.

### Embodiment 13

The method of any of embodiments 1-9, wherein the chemical fragment B is a methyl substituted pyridine compound.

### Embodiment 14

The method of any of embodiments 1-9, wherein the chemical fragment B includes a fused ring moiety selected from a quinoline, an isoquinoline, and an acridine.

### Embodiment 15

The method of any of embodiments 1-9, wherein the chemical fragment B has a formula selected from:

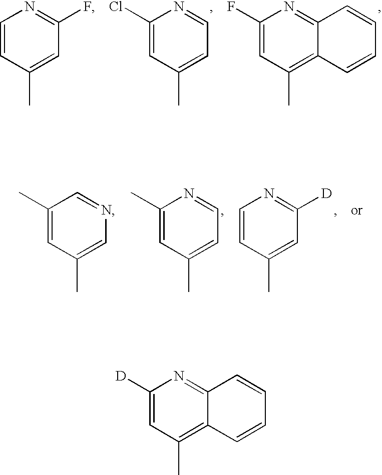

### Embodiment 16

The method of any of embodiments 1-15, wherein the alkylation reaction comprises: (i) reacting the chemical fragment A with a strong base and deprotonating the chemical fragment A at a carbon, oxygen, or sulfur atom; and (ii) reacting the deprotonated chemical fragment A with a methyl halide thereby methylating the chemical fragment A at the deprotonated atom.

### Embodiment 17

The method of any of embodiments 1-16, wherein the alkylation reaction of step (d) comprises: (i) reacting the chemical fragment A with a strong base and deprotonating the chemical fragment A at a carbon, oxygen, or sulfur atom; (ii) halogenating the methyl group of the chemical fragment B to obtain a derivative of chemical fragment B having a halogenated methyl group; and (iii) reacting the deprotonated chemical fragment A with the derivative of chemical fragment B having the halogenated methyl group, thereby forming a C—C, C—O, or C—S bond between the deprotonated carbon, oxygen, or sulfur atom, respectively, of the chemical fragment A and the methyl group carbon of the chemical fragment B.

### Embodiment 18

The method of embodiment 17, wherein halogenating is performed by reacting the chemical fragment B with N-bromosuccinimide (NBS) or N-chlorosuccinimide (NCS).

### Embodiment 19

A method for creating a chemical compound, namely A-B, from two chemical fragments, namely A and B, wherein the chemical compound binds to a KCNQ (Kv7) channel protein, the method comprising: (a) methylating one of the chemical fragments, A, at one or more positions to obtain a ¹³CH₃-methylated analog of A, namely A-¹³CH₃, by performing an alkylation reaction, wherein the di-methylated form of A-¹³CH₃ has a formula selected from:

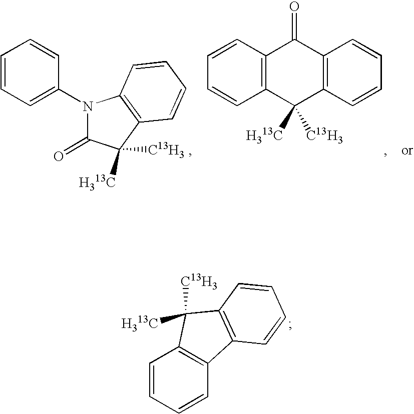

(b) forming a mixture comprising: (1) A-¹³CH₃; (2) the other chemical fragment, B, which is selected from compounds listed in Table 2 or 3, and (3) the KCNQ (Kv7) channel protein; (c) determining whether both A-¹³CH₃ and B bind to the target protein in the mixture such that the methyl group of A-¹³CH₃ and the methyl group of B are located no more than 5 angstroms apart; and if so (d) performing the alkylation reaction of step (a) using A and B as reagents (e.g., after B has been halogenated on its allylic or benzylic methyl group) in order to covalently attached A and B via the methyl group carbon atom of B to obtain the chemical compound A-B.

### Embodiment 20

A kit for use in any of embodiments 1-19, the kit comprising (a) a first chemical compound suitable for use as the chemical fragment A; (b) a second chemical compound suitable for use as the chemical fragment B; (optionally) (c) a methylating reagent comprising a ¹³CH₃-methyl group for methylating fragment A; and optionally (d) a halogenating agent for halogenating chemical fragment A and/or chemical fragment B.

### EXAMPLES

The following examples are illustrative and not intended to limit the claimed subject matter.

### Example 1

### NMR-Based Fragment Assembly Method

NMR-based fragment assembly has been described in the art. Reference is made to Sem D S. (1999) NMR-SOLVE Method for Rapid Ident. of Bi-Ligand Drug. U.S. Pat. No. 6,333,149 B1; Sem D S, Yu L, Coutts S M, and Jack. R. (2001) An Object-oriented Approach to Drug Design Enabled by NMR SOLVE, the First Real-Time Structural Tool for Characterizing Protein-Ligand Interactions. J. Cellular Biochemistry 37, S99-105; Sem D S, Pellecchia M, Dong Q, Kelly M, Lee M S (2003) NMR Assembly of Chemical Entities. US Publication No. 20030113751 A1; Sem D S, Bertolaet B, Baker B, Chang E, Costache A, Coutts S, Dong Q, Hansen M, Hong V, Huang X, Jack R M, Kho R, Lang H, Meininger D, Pellecchia M, Pierre F, Villar H, Yu L. (2004) Systems-based design of bi-ligand inhibitors of oxidoreductases: filling the chemical proteomic toolbox. Chem. Biol. 11, 185-194; and Sem D S (2006) Fragment-based Approaches in Drug Discovery (Jahnke and Erlanson, Ed.), pp 163-196; the contents of which are incorporated herein by reference in their entireties.

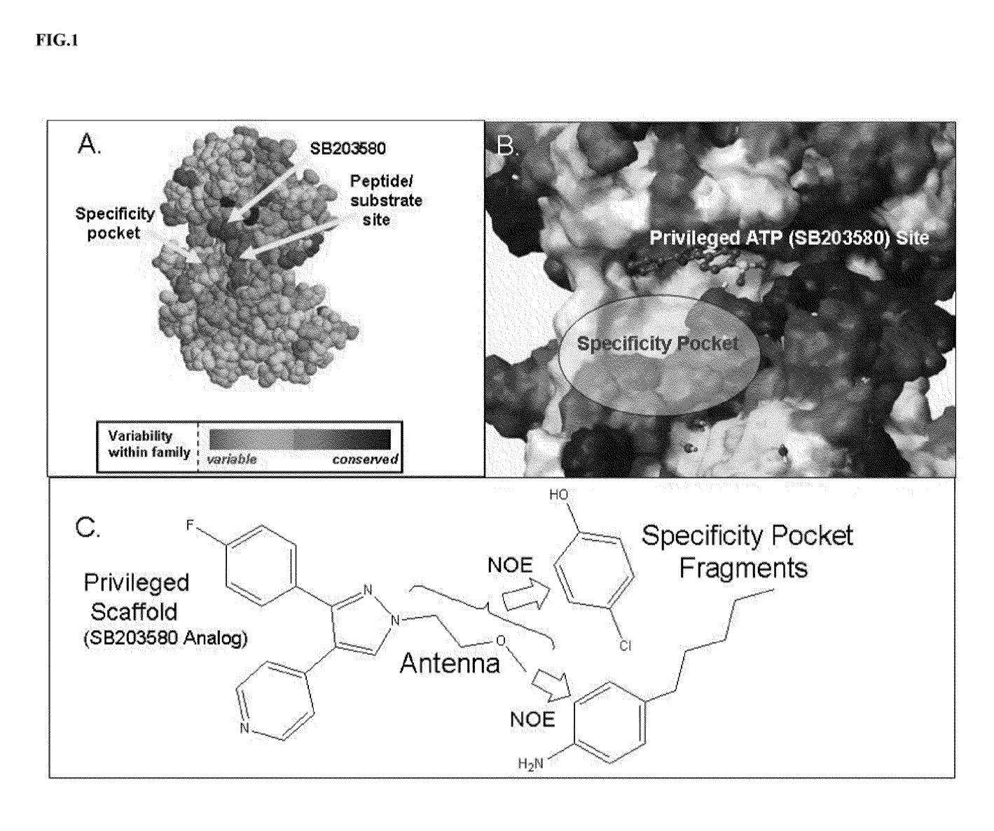

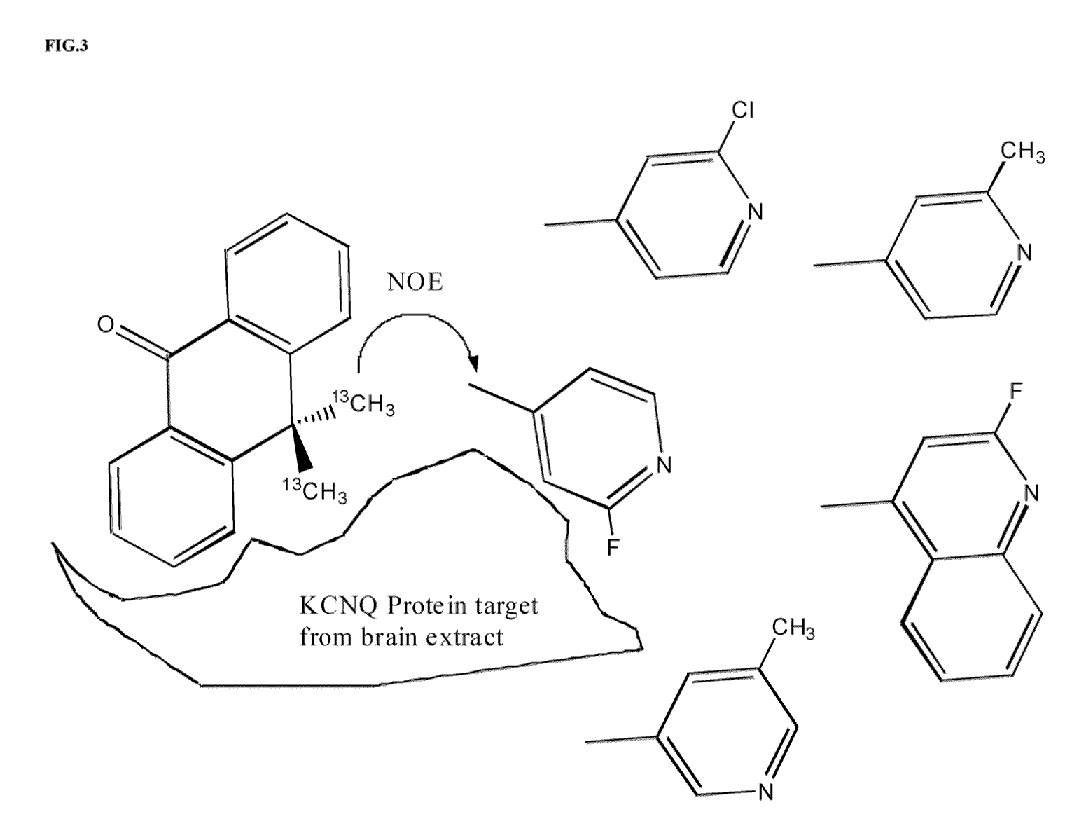

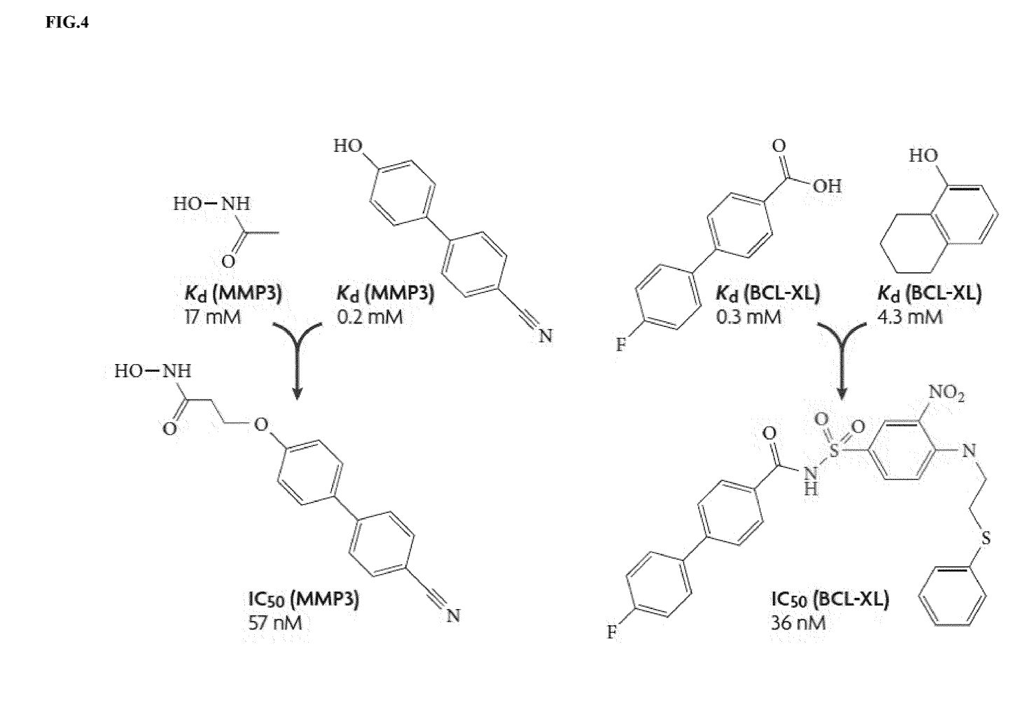

General fragment assembly methods may be illustrated here using example proteins referred to as p38α MAP kinase or KCNQ channel protein. A low concentration of the target protein (for example, 2-200 μM, although preferably 20-50 μM) is mixed with chemical fragments (e.g., heterocyclic ring structures of size ≦400 g/mol, and preferably ≦350 g/mol), and transfer of magnetization between the fragments (typically present at 0.2-20 mM) is measured. This “transfer”, termed an NOE (Nuclear Overhauser Effect), only occurs if both chemical fragments bind to the protein (p38α, MAPK or KCNQ as described below). Further, if an NOE is observed between two atoms, as indicated in FIG. 1 and in FIG. 3, it suggests that the two atoms are located in close proximity, because NOEs are only observed up to 5 Å (and intensity drops off as 1/(distance)⁶). Having observed an NOE, the two fragments may be chemically tethered at positions close to where the NOE was observed. This linkage produces a tremendous increase in affinity for the protein targets, because of the entropic advantage of binding only one (tethered) ligand, versus two (untethered) ligands as in FIG. 4. This effect is well-established (Shuker et al., 1996; Sem et al., 2004; Pellecchia et al., 2002; Sem, 2006), and one typically observes decreases in K_{d} (or IC₅₀) values of 1000-fold or more (e.g., 10 μM to 10 nM) due to linkage as in FIG. 4. The fragment assembly approach also identifies which two fragments will yield a high affinity ligand when tethered, before actually needing to synthesize the compound. This decreases much of the very time-consuming and expensive process of medicinal chemistry optimization that is needed to get to a final drug lead. For example, one could use the NMR-based fragment assembly method to screen 4×250 (=1,000) combinations of chemical fragment pairs (core=A×scaffold=B), and use the NMR method (e.g. NOE measurements) to identify those combinations that bind proximal to each other (i.e. within 5 angstroms). Using an estimated “hit rate” on the order of about 2%, about 20 combinations out of these 1,000 combinations may be selected and combined. Subsequently, the compound thereby, formed may be further tested in a binding assay (e.g., chemical proteomic assay using an affinity column) or a biological assay.

As shown in FIG. 4, chemical linkage of two weak binding fragments led to a new tethered fragment with much higher affinity for the protein drug target. (See Hajduk and Greer, Nature Reviews—Drug Discovery, Vol. 6, March 2007, 211-219). However, unlike the methods presented as part of this invention, the strategy shown required more involved chemical synthetic strategies to ultimately link fragment A and B. The example on the right side of FIG. 4 shows that additional chemical modifications may be required in order to make the final drug molecule

The disclosed methods can be applied to design inhibitors (i.e., “protein ligands” or “drug lead molecules”) for a wide range of protein drug targets. As an example, the KCNQ potassium ion channel may be utilized. The KCNQ ion channel is a therapeutic target for a variety of psychiatric disorders or CNS diseases. The present methods may be utilized to optimize or derivatize drugs existing drugs, such as those listed in Tables 4-6. Suitable drugs for the present methods may include drugs that have been through clinical trials for a CNS disease, and as such, are already known to be safe, bioavailable and able to cross the blood-brain barrier. Re-engineering of a drug used to treat one disease, so that it is now effective for a different disease, is called “repurposing.” Repurposing and methods for performing repurposing have been described. (See, e.g., Chong and Sullivan, Nature, Vol. 448, 9 Aug. 2007, 645-646; and Keiser et al., Nature, Vol. 462, 12 Nov. 2009, 175-182, the contents of which are incorporated herein by reference in their entireties). The methods described herein may be used for repurposing drugs, but can also be used to improve existing drugs for their intended purpose based on binding to their intended protein drug target. For example, the present methods may be utilized to derivatize an existing drug in order to increase affinity or specificity for binding to the intended protein drug target. The NMR fragment assembly methods being presented herein will guide changes to proven scaffold or core molecules (i.e. an important piece or fragment of the drug lead, which is conserved in medicinal chemistry SAR (structure-activity-relationship” studies)) for KCNQ-based drug leads, but in a unique manner that considers downstream synthetic strategy by using NMR probe groups (e.g., CH₃ reporter groups, that can be used to measure NOEs) that are attached to scaffold and pendant group fragment molecules using the same chemistry that will eventually be used to link scaffold and pendant groups. A drug or fragment thereof may be derivatized using the methods disclosed herein by identifying a drug or fragment having a nucleophilic carbon, oxygen, or sulfur atom and then using the drug or fragment as “chemical fragment A” in the methods disclosed herein.

The disclosed methods can be used to quickly optimize address potency, selectivity, or side-effect problems of an existing drug. As an example, a drug (e.g., DMP543) is chemically broken up into component fragments (A-B to A and B), for example where one fragment contains a nucleophilic carbon, oxygen, or sulfur atom and preferably where the one fragment is utilized in a synthesis method for the drug molecule. In some embodiments where fragment A has a nucleophilic carbon, fragment A has a formula:

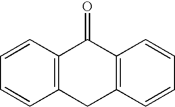

and fragment B has a formula:

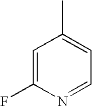

NMR-fragment assembly then is used to identify new suitable fragments to substitute for the original fragment B. New fragments are chosen based on their having similar pharmacophore features (e.g. hydrogen bond donor or acceptor atoms or hydrophobic groups) to the original fragment, with subtle addition of new features (e.g. additional donor or acceptor atoms, or increasing length of an aliphatic group)). In general, fragments should have molecular weight <400 g/mol (preferably <350 g/mol, and have ≦3 hydrogen bond donors or acceptors.

in order to facilitate later tethering to fragment A, fragment B preferably has an allylic or benzylic methyl group to permit chlorination with NCS, N-chlorosuccinimide or bromonation with NBS, N-bromosuccinimide. For example, in FIG. 1 a variant of a non-specific kinase inhibitor (drug lead molecule) from Smithkline Beecham (SB203580) was fragmented, and an NMR reporter group (called the “antenna”) was added, and new fragments were identified that bind close to the antenna atoms, and when these fragments were tethered to the scaffold, high affinity inhibitors were obtained that were selective for p38α MAP kinase. However, the fragments utilized in that method had no allylic or benzylic methyl groups to facilitate linkage and a complicated organic synthesis method was required to link the fragments. A ligand for KCNQ may be identified much more efficiently using the presently disclosed methods because fragment A and fragment B can be linked relatively easily after determining via NMR NOE analysis that fragment A and fragment B should be linked.

A significant disadvantage of NMR-fragment assembly methods of the prior art is that once it is established that two fragments are close, and should therefore be chemically joined, it is often not chemically possible to tether them, or it is chemically difficult and involves multiple synthetic steps. The methods disclosed herein address this problem, because the chemical reaction used to introduce the NMR probe (the ¹³C-methyl group attached to the nucleophilic atom of fragment A) for the NMR-NOE may subsequently be used to join the A and B fragments. The chemical fragment B is selected to contain an allylic or benzylic methyl group because such groups are easily and specifically halogenated so that the nucleophilic atom of chemical fragment A can attack the halogenated methyl group of chemical fragment B and displace the halogen to form a bond.

The above-described NMR fragment assembly methods may be utilized to identify ligands for the KCNQ potassium channel, which can be affinity-purified from rat brain extracts using an affinity column with ligands such as DMP543, XE991 or linopirdine, covalently attached to a resin. The KCNQ channel is a membrane-bound protein and is considered large for NMR studies. But, NOE and STD (saturation transfer difference) (Sem, 2006; Mayer and Meyer, 2001; Yao and Sem, 2001) based methods for measuring proximity of two fragments (or a fragment and a protein binding site) have been shown to work effectively even with very high molecular weight systems (Assadi-Porter et al., 2008) like membrane-bound KCNQ, especially (as in this case) when fragment binding will be in fast exchange (=low affinity) and, therefore, detectable by the NMR technique. Indeed, such methods have been recently applied to G-protein coupled receptors by using difference spectra in order to remove potential spectral artifacts from NMR experiments from chemical fragments that penetrate the lipid layer (Assadi-Porter et al, 2008). An important variation to that procedure (and inter-ligand NOE studies, as in FIG. 1), which is employed as part of the presently disclosed methods is to chemically place a ¹³C labeled methyl group as an NMR reported group (the “NMR probe”), analogous to the antenna in FIG. 1. Then, an NOE experiment could be performed, that is a 1D variant of the typical ¹³C half-filtered 2D NOESY, which selectively measures only NOEs between a ¹³C-attached proton and all other protons within 5 Å, whether or not they are ¹³C attached (hence the term half filtered). These experiments can be done on a 400 MHz, 500 MHz, 600 MHz or higher field NMR spectrometer, ideally equipped with a cryoprobe (and cryocooled ¹³C preamp). The fragment screening strategy presented herein could rely on established scaffolds (A fragments), from the DMP543 compound that was reported previously (Zaczek et al., 1998; Earl et al., 1998; Pest et al., 2000). It is noteworthy that the reported synthesis of these drug leads (Earl et al., 1998), based on these scaffolds (A), relied on base catalyzed linkage to para-methylpyridyl pendant (B) groups (after the methyl was halogenated with NCS, N-chlorosuccinimide), by attack of the scaffold carbanion on the —CH₂I group on the pendant group. That is, the synthesis method used to make this drug utilized an intermediate with a nucleophilic carbon, oxygen, or sulfur atom, making it a suitable fragment for use as chemical fragment A in the present methods.

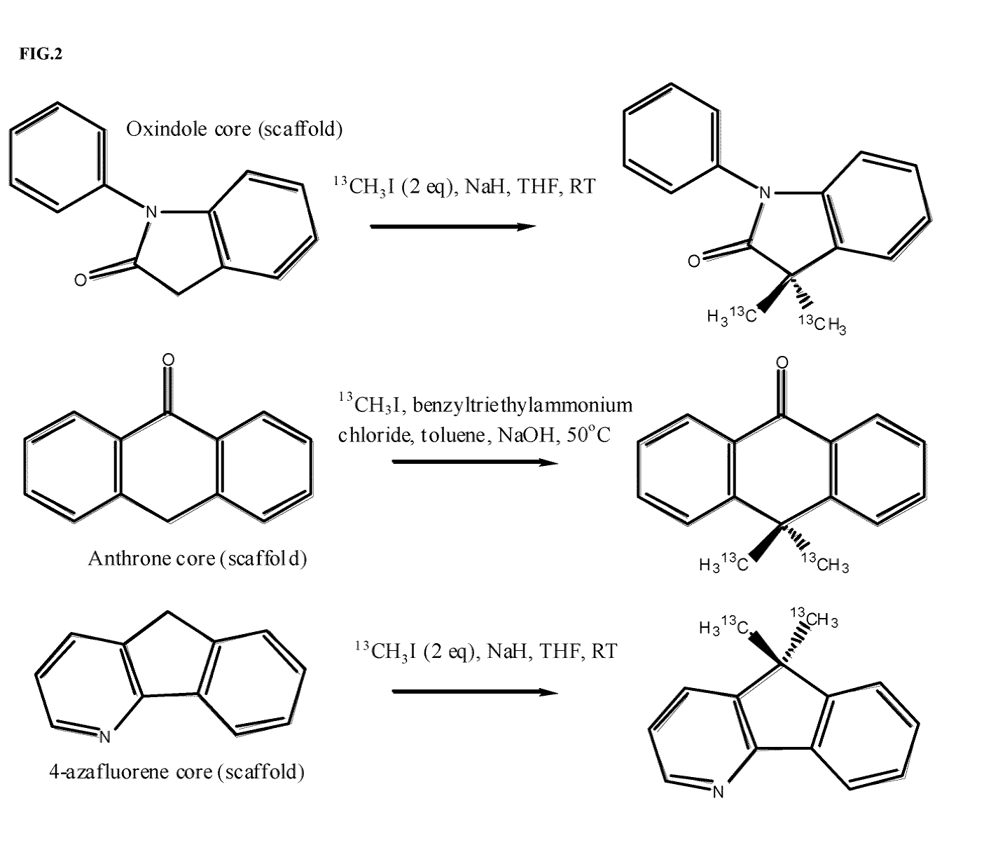

A feature of the present methods is the use of the same chemistry to introduce a ¹³C-labeled NMR reporter group (a methyl group) to a chemical fragment, A, for NMR-NOE analysis, as will be used to join the chemical fragment A, to a second chemical B. An example of one such chemical reaction is shown in FIG. 2. In this embodiment, the syntheses involve treatment with strong base to form the carbanion nucleophile, which then attacks the alkyl halide to give the methylated product. By controlling stoichiometry, it is possible to incorporate either one or two methyl group probes. These fragments were identified in the synthetic scheme for existing drugs DMP543, XE991 and linopirdine, based on steps where a carbanion intermediate occurred in the synthesis, but was used to attack a different electrophile (other than CH₃—I). Analogous methylated fragment A's can be prepared from any drug, by examining the synthetic strategy used to prepare the drug and determining if in any step a carbanion (or RS⁻ or RO⁻) nucleophilic intermediate was used. Examples of drugs of interest include those in Tables 4-6.

In FIG. 2, the labeled scaffold molecules (A-¹³CH₃) are added to the KCNQ protein solution ([KCNQ]=2-20 μM), which could contain deuterated detergent/micelles (e.g. perdeutoro-dilaurolylphasphatidyl choline), as described previously for NMR studies of membrane-bound proteins (Yao et al., 2008)). In the methods, a library of para-methyl (or ortho- or meta-methyl)pyridyl compounds/fragments (for example, 1,000 fragments, available from Sigma/Aldrich) might be screened one at a time, or in pools (e.g., of 10), to identify those B fragments which have the p-methyl group (or other group, and possibly also meta or ortho substituted) proximal to the ¹³CH₃— scaffold group on A, based on the observation of an NOE in a 1D ¹³C half filtered {¹H-¹H} NMR NOE experiment (see FIG. 3). The experiment shown in FIG. 3 may be performed with either the mono- or di-methylated fragment. In the example of FIG. 3, only the 2-fluoro-4-methylpyridine fragment B binds within 5 angstroms, and can show an NOE signal.

As a control in these experiments, the measured NOE or saturation transfer signal might be of the sample (perhaps a tissue extract) that has had the protein target removed (KCNQ in this case), which could be done using an affinity column. This control experiment could then be subtracted from the same experiment done in the presence of protein target, as described recently (Assadi-Porter (2008) 130, 7212). However, the present methods differ from those of Assadi-Porter in that the chemical fragment B contains an allylic or benzylic methyl group to facilitate chemical linkage in the process used to form the A-B compound.

Once a proximal-binding scaffold/pyridyl fragment pair (A and B) is identified, based on the NMR assay, the pair is chemically tethered (to make A-B) using the same chemical reaction (nucleophilic substitution on an alkyl halide, in this case) that was used to attach the NMR probe (the ¹³CH₃-methyl reporter group), similar to adding pendant groups to the scaffolds (cores) as shown in FIG. 2 (Earl et al., 1998). In one embodiment, the methyl on the pyridyl pendant group may be iodinated (chlorinate using NCS, then replace chlorine with iodine using NaI in acetone). Then, analogous to the reactions in FIG. 2, the I—CH₂-pyridyl pendant group would be added to the scaffold in a base catalyzed nucleophilic substitution.

The position for the NMR ¹³CH₃— reporter group on the scaffold may be selected based on any of the following criteria:: (a) the site is known to be an effective linkage site, perhaps from previous medicinal chemistry (Zaczek et at, 1998; Earl et al., 1998; Pest et al., 2000); and (b) has a chemical attachment chemistry that is established and robust, so lends itself well to subsequent chemical tethering of the scaffold fragment and the newly identified pendant group fragment. One preferred reaction for linking the chemical fragments A and B is a substitution reaction, where a nucleophilic atom (e.g., C, O, or S) attacks an alkyl halide, such as a halogenated allylic methyl group or a halogenated benzylic methyl group. The NMR-based fragment screening and assembly presented here is designed so that subsequent chemical tethering can be done using a robust chemical reaction (e.g., a nucleophilic substitution on a primary carbon via an S_{N}2 reaction), which should take only a matter of days for a given scaffold/pendant group pair to go from NMR NOE result to synthesis of the A-B ligand. Because this method relies on existing molecules that bind to protein drug targets, it is especially well-suited to: (a) optimizing a current drug to be more potent for an intended target, and (b) re-engineering a drug to treat a different disease than was originally intended (i.e., repurposing).

In the above experiments, one could use any of a number of assays to determine whether the chemical fragments (A and B) and the chemical compound synthesized therefrom (A-B) bind to a target protein, including a chemical-proteomic type assay. For example, a binding assay may be performed as follows: (a) passing a biological sample including a target protein and a non-target protein over a first column, the column containing an affinity resin for the target protein, the affinity resin made of a resin conjugated to a first chemical compound (A-B); (b) washing the column and removing proteins that are not bound to the affinity resin; (c) eluting proteins from the column that are bound to the affinity resin; (d) identifying proteins in the eluate including the target protein and optionally the non-target protein. Such a method may be utilized to identify (e.g., based on patterns of bands in an SDS-PAGE gel of column eluate) a set of proteins in a sample from a target organ (e.g. brain) and a sample from an anti-target organ (e.g. heart muscle) that bind to the optimized drug molecules. Protein bands of interest can be identified using standard mass spectrometry methods, such as LC-MS/MS. Preferably, the methods identify an optimized drug lead(s) with increased specificity for an intended target, which is the KCNQ target protein in the example above—and this will be assessed based on the protein elution profile from an affinity resin, when the improved lead molecules are used. For example, an improved DMP543 drug lead (A-B*) might elute only KCNQ2-5 proteins from the column, but significantly fewer or no other off-target proteins that bound the original DMP543 molecule (A-B). The best molecules, as judged by the binding affinity to KCNQ channel in the brain tissue (e.g. using a competitive STD assay), lack of binding to the heart muscle KCNQ1/mink channel (which would produce dangerous side effects), and in general the lowest number of off-target binding events, could then be chosen for evaluation in subsequent animal model studies. Complementary behavioral assays, using the newly designed compounds would allow correlation of protein binding profiles with drug efficacy, as well as with undesired effects.

### Example 2

### Methylation of Anthrone

The following is a procedure for the preparation of 10-(Phenylalkyl)-9 (10H) anthracenone, incorporating the ¹³C methyl groups to make a A-¹³CH₃ fragment A (shown in FIGS. 2 and 3). 9(10H)-Anthracenone (1 g, 5.15 mmol) and dry K₂CO₃ (2 g) were suspended in absolute acetone (80 mL) under N₂. Methyl chloride (5.2 mmol) and catalytic amounts of potassium iodide (100 mg) were added (benzyl chloride may be substituted instead), and the mixture was refluxed under nitrogen until the reaction was completed (monitored with TLC, comparing reaction versus starting materials; solvent system=9:1 hexane:acetone). The reaction mixture was then cooled and poured into water (400 mL), acidified with 6 N HCl, and extracted with CH₂Cl₂ (3×30 mL). The combined CH₂Cl₂ extracts were washed, dried over Na₂SO₄, and then evaporated. The residue was purified by silica gel chromatography.

The above reaction was repeated, with slight modification, using the following amounts: 0.5 g of anthrone (0.00257 mol) and 0.368 g (=0.0162 ml neat solution=0.00257 mol) of ¹³CH₃I then this amount was doubled in the same reaction on the next day, as there was a big spot of the anthrone remaining on the TLC plate (indicating incomplete reaction). An additional 0.368 g of ¹³CH₃I was added to the reaction. In the separation step, the reaction mixture was purified using flash column chromatography, using an eluent of 97:3 hexane:acetone.

### Example 3

### Method Applied to Synthetic Intermediate for a Drug

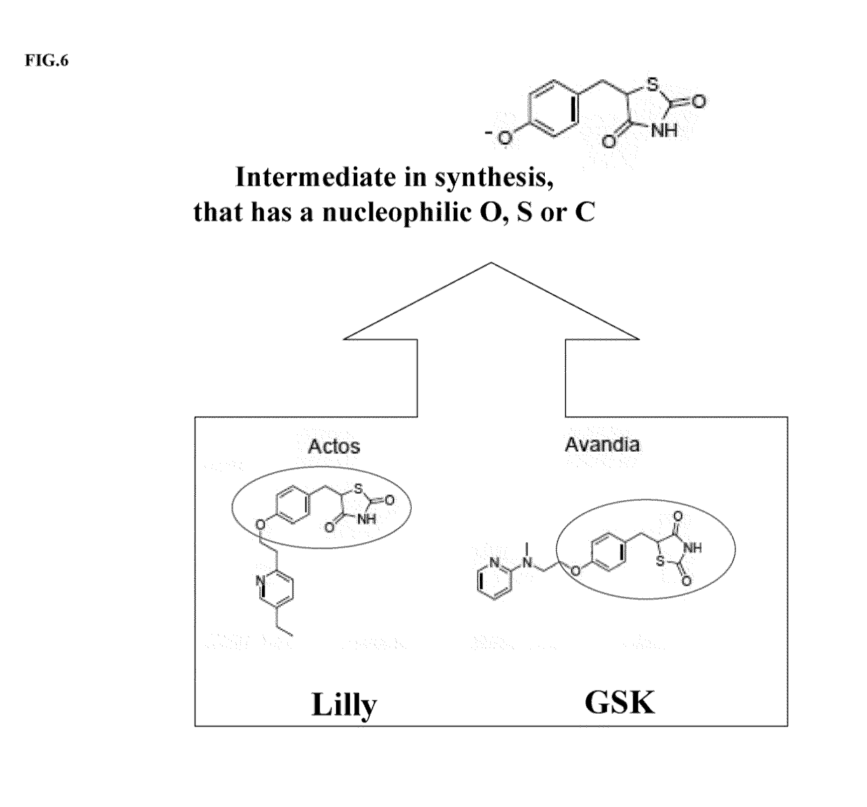

Two drugs, Avandia (GSK) and Actos (Lilly), both contain a common chemical core or scaffold called glitazone (FIG. 6). Other examples of such intermediates could be easily identified by surveying the synthetic procedures used to make existing drugs, such as those in Tables 4-6.

The chemical scaffold of glitazone includes a thiazolidinedione ring joined via a methylene to a phenol. The phenol oxygen of glitazone is chemically linked to two different pendant groups in the two different drugs. Glitazone is a synthetic intermediate on the pathway for synthesis of these two drugs, and it also possesses a nucleophilic atom (the phenolic oxygen), making it a suitable fragment A.

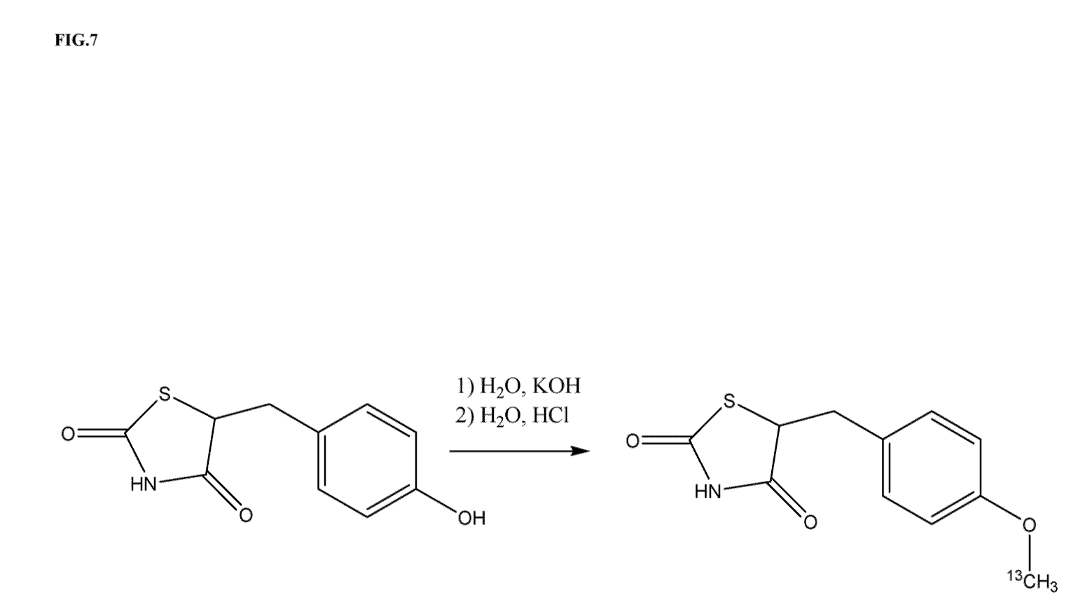

The phenolic oxygen of glitazone can be methylated be reacting with ¹³CH₃I in the presence of base to give the methyl ether, shown in FIG. 7, and a suitable A-¹³CH₃ fragment for the disclosed method. This fragment is then used to screen in the NMR assay for fragment B groups, as in FIG. 3, and when one is identified it is chemically linked to the halogenated fragment B, to give A-B.

Various B fragments can be chosen to make various A-B ligands, optimizing for a number of purposes. For example, there is a danger of heart attack associated with taking Avandia, so one optimization strategy could be to identify alternative fragment B's that bind preferentially to the target of the drug (which is the PPAR gamma protein) and less to non-target proteins from heart tissue. This would be an example of optimizing a drug to reduce side effects. Alternatively, one could identify all the proteins that bind to glitazone using a proteomic assay, and if one of the non-target proteins (e.g., an ion channel such as KCNQ) is the target for another disease, such as a psychiatric disorder, then alternative fragment B's could be identified to achieve higher binding affinity for the ion channel, relative to the target protein. This is an example of drug repurposing, where a drug originally designed to treat a first disease by virtue of preferred binding to a first protein target, is chemically modified to now treat a second disease by virtue of binding preferentially to a second protein target.

### REFERENCES

Assadi-Porter F M, Tonelli M, Maillet E, Hallenga K, Benard O, Max M, Markley J L (2008) Direct NMR detection of the binding of functional ligands to the human sweet receptor, a heterodimeric family 3 GPCR. J. Am. Chem. Soc. 130, 7212-7213.
Bakshi V P, Geyer M A (1997) Phencyclidine-induced deficits in prepulse inhibition of startle are blocked by prazosin, an alpha-1 noradrenergic antagonist. J. Pharmacol. Exp. Ther. 283, 666-674.
Brown D A, Adams P R (1980) Muscarinic suppression of a novel voltage-sensitive K+ current in a vertebrate neurone. Nature 283, 673-676.
Earl R A, Zaczek R, Teleha C A, Fisher B N, Maciag C M, Marynowski M E, Logue A R, Tam S W, Tinker W J, Huang S M, Chorvat R J. (1998) 2-Fluoro-4-pyridinylmethyl analogues of linopirdine as orally active acetylcholine release-enhancing agents with good efficacy and duration of action. J. Med. Chem. 41, 4615-4622.
Elmedyb P, Canoe K, Schmitt N, Hansen R S, Grunnet M, Olesen S P (2007) Modulation of ERG channels by XE991. Basic & Clinical Pharmacol. & Toxicol. 100, 316-322.
Fenton W S, McGlashan T H (1991) Natural history of schizophrenia subtypes. II. Positive and negative symptoms and long-term course. Arch. Gen. Psychiatry 48, 978-986.
Ge X, Wakim B, and Sem D S (2008) Chemical proteomics-based drug design: target and antitarget fishing with a catechol-rhodanine privileged scaffold for NAD(P)(H) binding proteins. J. Med. Chem., 51, 4571-4580.
Hajduk P J, and Greer J (2007) A decade of fragment-based drug design: strategic advances and lessons learned. Nature Reviews Drug Disc. 6, 211-219.
Lieberman J A, Stroup T S, McEvoy J P, Swartz M S, Rosenheck R A, Perkins D O, Keefe R S, Davis S M, Davis C E, Lebowitz B D, Severe J, Hsiao J K; Clinical Antipsychotic Trials of Intervention Effectiveness (CATIE) Investigators (2005) Effectiveness of antipsychotic drugs in patients with chronic schizophrenia. N. Engl. J. Med. 353, 1209-1223.
Mayer M, and Meyer B (2001) Group epitope mapping by saturation transfer difference NMR to identify segments of a ligand in direct contact with a protein receptor. J. Am. Chem. Soc. 123, 6108-6117.
Meyers B and Kritzer M F (2009) In vitro binding assays using (3)H nisoxetine and (3)H WIN 35,428 reveal selective effects of gonadectomy and hormone replacement in adult male rats on norepinephrine but not dopamine transporter sites in the cerebral corte.g. Neuroscience 159, 271-282.
Pellecchia M, Sem D S, and Wuthrich K. (2002) NMR in drug discovery. Nat. Rev. Drug Discov. 1, 211-219.
Pest J A, Huhn G F, Yin 3, Xing Y, Fortunak J M, and Earl R A (2000) Efficient pyridinylmethyl functionalization: synthesis of 10,10-Bis[(2-fluoro-4-pyridinyl)methyl]-9(10H)-anthracenone (DMP 543), an acetylcholine release enhancing agent. J. Org. Chem. 65, 7718-7722.
Peters E C, and Gray N S (2007) Chemical proteomics identifies unanticipated targets of clinical kinase inhibitors. ACS Chemical Biology 2, 661-664.
Saganich M J, Machado E, Rudy B (2001) Differential expression of genes encoding subthreshold-operating voltage-gated K+ channels in brain. J. Neurosci. 21, 4609-4624.
Schnee M E, Brown B S (1998) Selectivity of linopirdine (DuP 996), a neurotransmitter release enhancer, in blocking voltage-dependent and calcium-activated potassium currents in hippocampal neurons. J. Pharmacol. Exp. Ther. 286, 709-717.
Sem D S. (1999) NMR-SOLVE Method for Rapid Ident of Bi-Ligand Drug. U.S. Pat. No. 6,333,149 B1 ed., US (issued).
Sem D S, Yu L, Coutts S M, and Jack R. (2001) An Object-oriented Approach to Drug Design Enabled by NMR SOLVE, the First Real-Time Structural Tool for Characterizing Protein-Ligand Interactions. J. Cellular Biochemistry 37, S99-105.
Sem D S, Pellecchia M, Dong Q, Kelly M, Lee M S (2003) NMR Assembly of Chemical Entities. 20030113751 A1, US (pending).
Sem D S, Bertolaet B, Baker B, Chang E, Costache A, Coutts S, Dong Q, Hansen M, Hong V, Huang X, Jack R M, Kho R, Lang H, Meininger D, Pellecchia M, Pierre F, Villar H, Yu L. (2004) Systems-based design of bi-ligand inhibitors of oxidoreductases: filling the chemical proteomic toolbox. Chem. Biol. 11, 185-194.
Sem D S (2006) Fragment-based Approaches in Drug Discover (Jahnke and Erlanson, Ed.), pp 163-196.
Shuker S B, Hajduk P J, Meadows R P, Fesik S W (1996) Discovering high-affinity ligands for proteins: SAR by NMR. Science. 274, 1531-1534.
Sleno L, and Emili A (2008) Proteomic methods for drug target discovery. Curr. Opin. Chem. Biol. 12, 46.
Tam S W (1983) Naloxone-inaccessible sigma receptor in rat central nervous system. Proc. Natl. Acad. Sci. 80, 6703-6707.
Tam S W, Rominger D, Nickolson V J (1991) Novel receptor site involved in enhancement of stimulus-induced acetylcholine, dopamine, and serotonin release. (1991) Mol. Pharmacol. 40, 16-21.
Wang Q, Curran M E, Splawski I, Burn T C, Millholland J M, VanRaay T J, Shen J, Timothy K W, Vincent G M, de Jager T, Schwartz P J, Toubin J A, Moss A J, Atkinson
D L, Landes G M, Connors T D, Keating M T (1996) Positional cloning of a novel potassium channel gene: KVLQT1 mutations cause cardiac arrhythmias. Nat. Genet. 12, 17-23.
Wang H S, Brown B S, McKinnon D, Cohen I S (2000) Molecular basis for differential sensitivity of KCNQ and I(Ks) channels to the cognitive enhancer XE991. Mol. Pharmacol. 57, 218-1223.
Wang H S, Pan Z, Shi W, Brown B S, Wymore R S, Cohen I S, Dixon J E, McKinnon D (1998) KCNQ2 and KCNQ3 potassium channel subunits: molecular correlates of the M-channel. Science 282, 1890-1983.
Yao H, and Sem D S (2005) Cofactor fingerprinting with STD NMR to characterize proteins of unknown function: identification of a rare cCMP cofactor preference. FEBS Letters 579, 661-666.
Yao H, Stuart R, Cai S, and Sem, D S (2008) Structural characterization of the transmembrane domain from Subunit e (Su e) of yeast F₁F₀-ATP synthase: a helical GXXXG motif located just under the micelle surface. Biochemistry 47, 1910-1917.
Zaczek R, Chorvat R J, Saye J A, Pierdomenico M E, Maciag C M, Logue A R, Fisher B N, Rominger D H, Earl R A (1998) Two new potent neurotransmitter release enhancers, 10,10-bis(4-pyridinylmethyl)-9(10H)-anthracenone and 10,10-bis(2-fluoro-4-pyridinylmethyl)-9(10H)-anthracenone: comparison to linopirdine. J. Pharmacol. Exp. Ther. 285, 724-730.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

TABLE 1
Exemplary list of thiol compounds available from Chemical Proteomics Facility of
Marquette University at its website (accessed Jun. 1, 2010).

| | | | | |
|---|---|---|---|---|
| 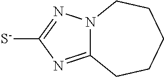 | 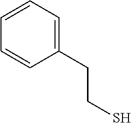 | 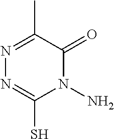 | 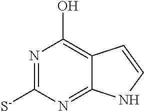 | 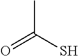 |
| 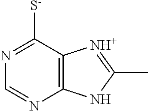 | 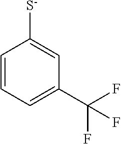 | 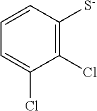 | 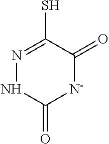 | 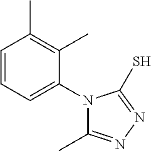 |
| 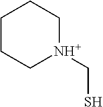 | 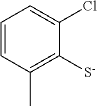 | 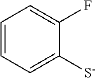 | 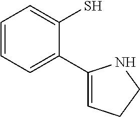 | 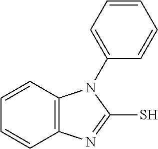 |
| 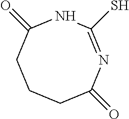 | 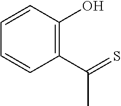 | 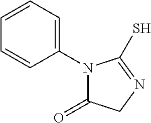 | 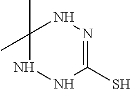 | 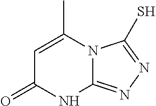 |
| 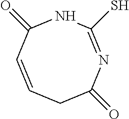 | 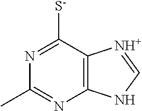 | 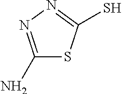 | 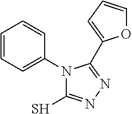 | 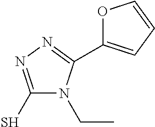 |
| 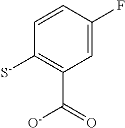 | 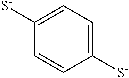 | 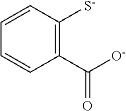 | 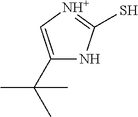 | 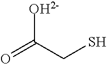 |
| 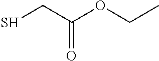 | 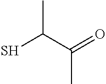 | 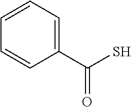 | 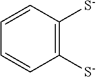 | 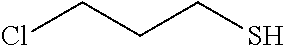 |
| 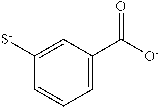 | 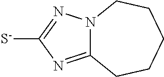 | 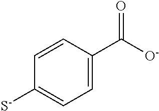 | 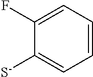 | 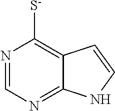 |
| 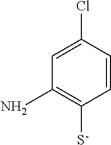 | 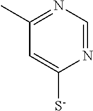 | 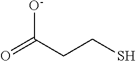 | 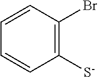 | 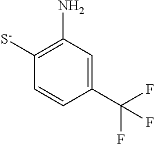 |
| 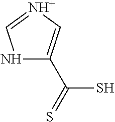 | 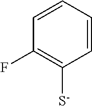 | 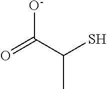 | 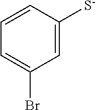 | 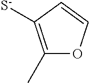 |
| 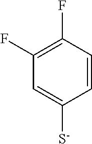 | 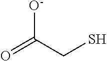 | 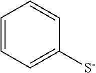 | 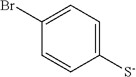 | 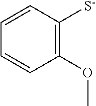 |
| 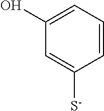 | 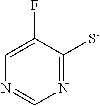 | 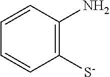 | 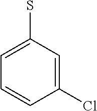 | 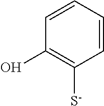 |
| 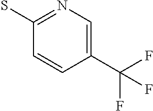 | 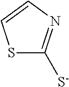 | 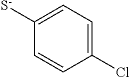 | 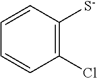 | 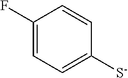 |
| 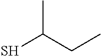 | 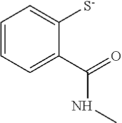 | 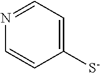 | 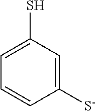 | 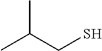 |
| 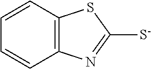 | 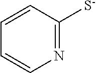 | 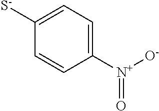 | 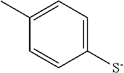 | 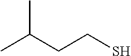 |
| 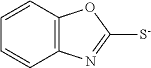 | 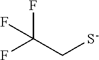 | 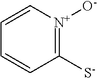 | 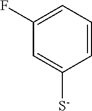 | 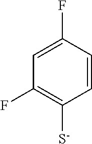 |
| 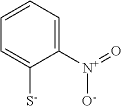 | 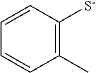 | 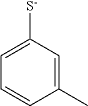 | 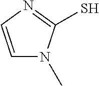 | 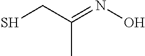 |
| 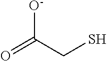 | 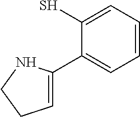 | 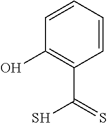 | 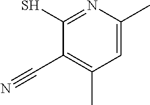 | 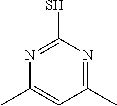 |
| 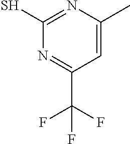 | 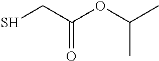 | 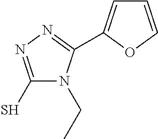 | 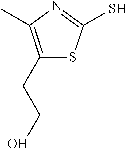 | 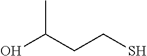 |
| 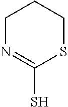 | 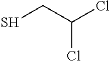 | 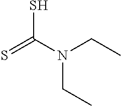 | 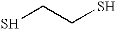 | 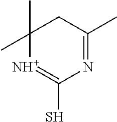 |
| 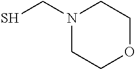 | 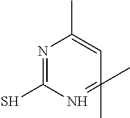 | 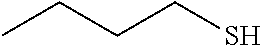 | 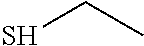 | 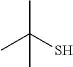 |
| 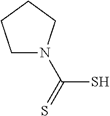 | 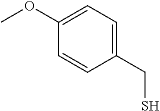 | 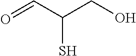 | 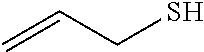 | 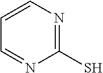 |
| 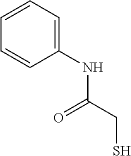 | 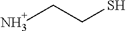 | 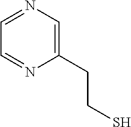 | 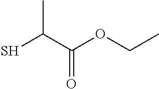 | 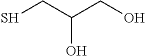 |
| 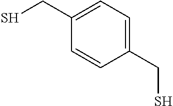 | 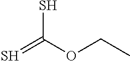 | 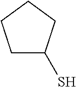 | 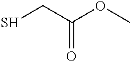 | 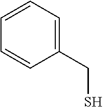 |
| 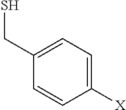 | 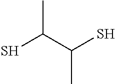 | 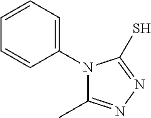 | 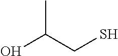 | 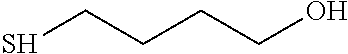 |
| 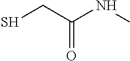 | 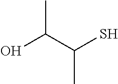 | 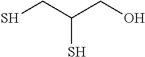 | 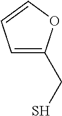 | 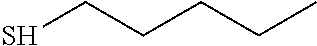 |
| 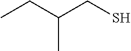 | 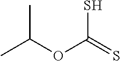 | 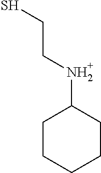 | 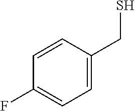 | 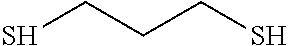 |
| 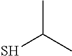 | 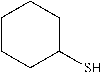 | 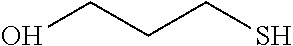 | 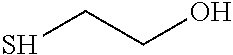 | 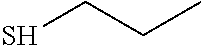 |

TABLE 2

| | |
|---|---|
| 5,6-Dimethylbenzimidazole | 1-M-TOLYL-PIPERAZINE, DIHYDROCHLORIDE |
| 5-Methylbenzimidazole | 1-(o-Tolyl)piperazine hydrochloride |
| 2-Amino-5,6-dimethylbenzimidazole | 1-(2,3-Xylyl)piperazine monohydrochloride |
| 1-PIPERIDINO-1-ISOBUTENE | 5-AMINO-3-METHYL-1-(P-TOLYL)PYRAZOLE |
| 7-Methylindole | 3-Methyl-1-(2-methylphenyl)-1H-pyrazol-5-amine |
| 6-Methylindole | 6-Methyl-5-nitroquinoline |
| 5-Methyltryptamine hydrochloride | 2,6-Dimethylquinoline |
| 5-Methoxy-4-methylindole | 8-Methylquinoline |
| 2,5-Dimethylindole | 6-Methylquinoline |
| 1-(p-Tolylsulfonyl)pyrrole | 7-Methylquinoline |
| 7-Methyltryptamine | 5,7-Dimethyl-8-quinolinol |
| 5-Methylindole | 2,7-DIMETHYLQUINOLINE |
| Tricyclazol | 5-Methyl-1,10-phenanthroline |
| 2,5-DIMETHYLBENZOTHIAZOLE | 5-Amino-6-methylquinoline |
| 2-HYDROXY-3,6,7-TRIMETHYLQUINOXALINE | 4-Hydroxy-2,6-dimethylquinoline |
| 5-Methylquinoxaline | 1-AMINO-2-METHYLNAPHTHALENE |
| | HYDROCHLORIDE |
| (−)-Isopulegol | 4-METHYL-1-NAPHTHALENEMETHANOL |
| (+)-Isopulegol | 2-Methyl-1-naphthol |
| (+)-Dihydrocarveol | 1-(3-Methylbenzyl)piperazine |
| (−)-Dihydrocarveol | 1-(2-Methylbenzyl)piperazine |
| DIHYDROCARVEOL | 1-(4-Methylbenzyl)piperazine |
| 5-(P-TOLYL)ISOXAZOLE | 1-(2,4,6-Trimethylbenzyl)piperazine |
| 2-(5-Isoxazolyl)-4-methylphenol | 5-Amino-3-(4-methylphenyl)pyrazole |

TABLE 3

| Compound | Formula |
|---|---|
| 1 | 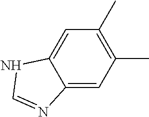 |
| 2 | 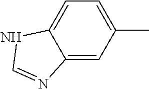 |
| 3 | 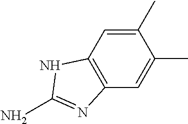 |
| 4 | 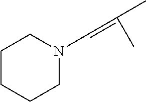 |
| 5 | 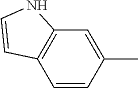 |
| 6 | 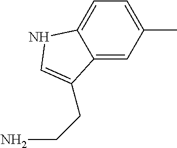 |
| 7 | 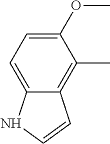 |
| 8 | 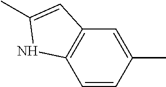 |
| 9 | 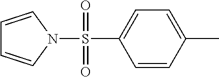 |
| 10 | 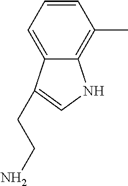 |
| 11 | 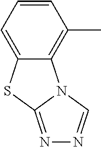 |
| 12 | 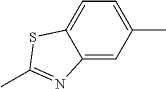 |
| 13 | 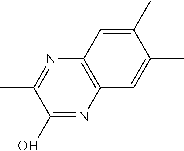 |
| 14 | 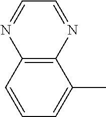 |
| 15 | 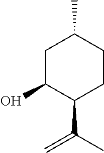 |
| 16 | 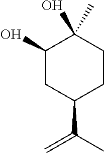 |
| 17 | 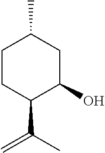 |
| 18 | 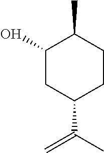 |
| 19 | 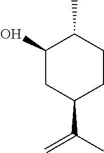 |
| 20 | 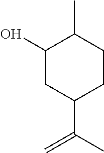 |
| 21 | 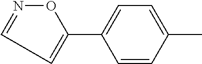 |
| 22 | 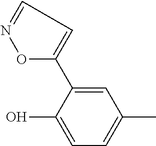 |
| 23 | 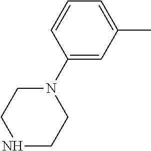 |
| 24 | 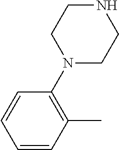 |
| 25 | 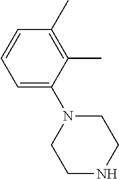 |
| 26 | 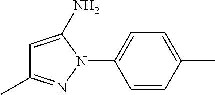 |
| 27 | 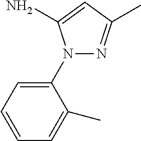 |
| 28 | 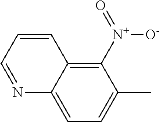 |
| 29 | 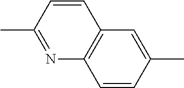 |
| 30 | 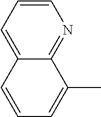 |
| 31 | 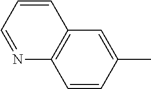 |
| 32 | 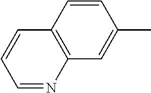 |
| 33 | 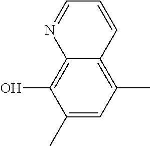 |
| 34 | 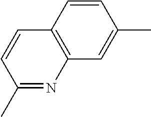 |
| 35 | 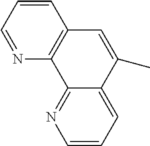 |
| 36 | 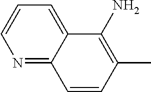 |
| 37 | 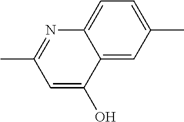 |
| 38 | 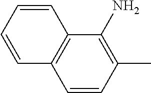 |
| 39 | 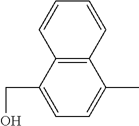 |
| 40 | 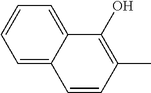 |
| 41 | 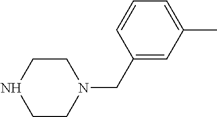 |
| 42 | 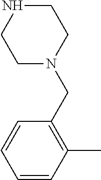 |
| 43 | 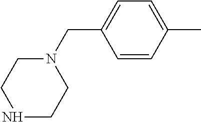 |
| 44 | 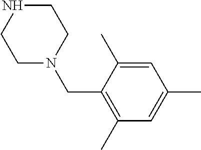 |
| 45 | 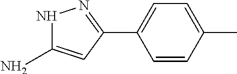 |

TABLE 4

| | | |
|---|---|---|
| Azaguanine-8 | Primaquine diphosphate | Torsemide |
| Allantoin | Progesterone | Halofantrine hydrochloride |
| Acetazolamide | Felodipine | Articaine hydrochloride |
| Metformin hydrochloride | Serotonin hydrochloride | Nomegestrol acetate |
| Atracurium besylate | Cefotiam hydrochloride | Pancuronium bromide |
| Isoflupredone acetate | Benperidol | Molindone hydrochloride |
| Amiloride hydrochloride dihydrate | Cefaclor | Alcuronium chloride |
| Amprolium hydrochloride | Colistin sulfate | Zalcitabine |
| Hydrochlorothiazide | Daunorubicin hydrochloride | Methyldopate hydrochloride |
| Sulfaguanidine | Dosulepin hydrochloride | Levocabastine hydrochloride |
| Meticrane | Ceftazidime pentahydrate | Pyrvinium pamoate |
| Benzonatate | Iobenguane sulfate | Etomidate |
| Hydroflumethiazide | Metixene hydrochloride | Tridihexethyl chloride |
| Sulfacetamide sodic hydrate | Nitrofural | Penbutolol sulfate |
| Heptaminol hydrochloride | Omeprazole | Prednicarbate |
| Sulfathiazole | Propylthiouracil | Sertaconazole nitrate |
| Levodopa | Terconazole | Repaglinide |
| Idoxuridine | Tiaprofenic acid | Piretanide |
| Captopril | Vancomycin hydrochloride | Piperacetazine |
| Minoxidil | Artemisinin | Oxyphenbutazone |
| Sulfaphenazole | Propafenone hydrochloride | Quinethazone |
| Panthenol (D) | Ethamivan | Moricizine hydrochloride |
| Sulfadiazine | Vigabatrin | Iopanoic acid |
| Norethynodrel | Biperiden hydrochloride | Pivmecillinam hydrochloride |
| Thiamphenicol | Cetirizine dihydrochloride | Levopropoxyphene napsylate |
| Cimetidine | Etifenin | Piperidolate hydrochloride |
| Doxylamine succinate | Metaproterenol sulfate, | Trifluridine |
| | orciprenaline sulfate | |
| Ethambutol dihydrochloride | Sisomicin sulfate | Oxprenolol hydrochloride |
| Antipyrine | Resveratrol | Ondansetron Hydrochloride |
| Antipyrine, 4-hydroxy | Bromperidol | Propoxycaine hydrochloride |
| Chloramphenicol | Cyclizine hydrochloride | Oxaprozin |
| Epirizole | Fluoxetine hydrochloride | Phensuximide |
| Diprophylline | Iohexol | Ioxaglic acid |
| Triamterene | Norcyclobenzaprine | Naftifine hydrochloride |
| Dapsone | Pyrazinamide | Meprylcaine hydrochloride |
| Troleandomycin | Trimethadione | Milrinone |
| Pyrimethamine | Lovastatin | Methantheline bromide |
| Hexamethonium dibromide dihydrate | Nystatine | Ticarcillin sodium |
| Diflunisal | Budesonide | Thiethylperazine malate |
| Niclosamide | Imipenem | Mesalamine |
| Procaine hydrochloride | Sulfasalazine | Imidurea |
| Moxisylyte hydrochoride | Thiostrepton | Lansoprazole |
| Betazole hydrochloride | Tiabendazole | Bethanechol chloride |
| Isoxicam | Rifampicin | Cyproterone acetate |
| Naproxen | Ethionamide | (R)-Propranolol hydrochloride |
| Naphazoline hydrochloride | Tenoxicam | Ciprofibrate |
| Ticlopidine hydrochloride | Triflusal | Benzylpenicillin sodium |
| Dicyclomine hydrochloride | Mesoridazine besylate | Chlorambucil |
| Amyleine hydrochloride | Trolox | Methiazole |
| Lidocaine hydrochloride | Pirenperone | (S)-propranolol hydrochloride |
| Trichlorfon | Isoquinoline, 6,7-dimethoxy-1- | (−)-Eseroline fumarate salt |
| | methyl-1,2,3,4-tetrahydro, | |
| | hydrochloride | |
| Carbamazepine | Phenacetin | Leucomisine |
| Triflupromazine hydrochloride | Atovaquone | D-cycloserine |
| Mefenamic acid | Methoxamine hydrochloride | 2-Chloropyrazine |
| Acetohexamide | (R)-(+)-Atenolol | (+,−)-Synephrine |
| Sulpiride | Piracetam | (S)-(−)-Cycloserine |
| Benoxinate hydrochloride | Phenindione | Homosalate |
| Oxethazaine | Thiocolchicoside | Spaglumic acid |
| Pheniramine maleate | Clorsulon | Ranolazine |
| Tolazoline hydrochloride | Ciclopirox ethanolamine | Sulfadoxine |
| Morantel tartrate | Probenecid | Cyclopentolate hydrochloride |
| Homatropine hydrobromide (R,S) | Betahistine mesylate | Estriol |
| Nifedipine | Tobramycin | (−)-Isoproterenol hydrochloride |
| Chlorpromazine hydrochloride | Tetramisole hydrochloride | Nialamide |
| Diphenhydramine hydrochloride | Pregnenolone | Perindopril |
| Minaprine dihydrochloride | Molsidomine | Fexofenadine HCl |
| Miconazole | Chloroquine diphosphate | Clonixin Lysinate |
| Isoxsuprine hydrochloride | Trimetazidine dihydrochloride | Verteporfin |
| Acebutolol hydrochloride | Parthenolide | Meropenem |
| Tolnaftate | Hexetidine | Ramipril |
| Todralazine hydrochloride | Selegiline hydrochloride | Mephenytoin |
| Imipramine hydrochloride | Pentamidine isethionate | Rifabutin |
| Sulindac | Tolazamide | Parbendazole |
| Amitryptiline hydrochloride | Nifuroxazide | Mecamylamine hydrochloride |
| Adiphenine hydrochloride | Dirithromycin | Procarbazine hydrochloride |
| Dibucaine | Gliclazide | Viomycin sulfate |
| Prednisone | DO 897/99 | Saquinavir mesylate |
| Thioridazine hydrochloride | Prenylamine lactate | Ronidazole |
| Diphemanil methylsulfate | Atropine sulfate monohydrate | Dorzolamide hydrochloride |
| Trimethobenzamide hydrochloride | Eserine sulfate, physostigmine | Azaperone |
| | sulfate | |
| Metronidazole | Tetracaine hydrochloride | Cefepime hydrochloride |
| Edrophonium chloride | Mometasone furoate | Clocortolone pivalate |
| Moroxidine hydrochloride | Dacarbazine | Nadifloxacin |
| Baclofen (R,S) | Acetopromazine maleate salt | Carbadox |
| Acyclovir | Lobelanidine hydrochloride | Oxiconazole Nitrate |
| Diazoxide | Papaverine hydrochloride | Acipimox |
| Amidopyrine | Yohimbine hydrochloride | Benazepril HCl |
| Pindolol | Lobeline alpha (−) hydrochoride | Azelastine HCl |
| Khellin | Cilostazol | Celiprolol HCl |
| Zimelidine dihydrochloride | Galanthamine hydrobromide | Cytarabine |
| monohydrate | | |
| Azacyclonol | Diclofenac sodium | Doxofylline |
| Azathioprine | Convolamine hydrochloride | Esmolol hydrochloride |
| Lynestrenol | Xylazine | Itraconazole |
| Guanabenz acetate | Eburnamonine (−) | Liranaftate |
| Disulfiram | Harmaline hydrochloride dihydrate | Mirtazapine |
| Acetylsalicylsalicylic acid | Harmalol hydrochloride dihydrate | Modafinil |
| Mianserine hydrochloride | Harmol hydrochloride monohydrate | Nefazodone HCl |
| Nocodazole | Harmine hydrochloride | Nilvadipine |
| R(−) Apomorphine hydrochloride | Chrysene-1,4-quinone | Oxcarbazepine |
| hemihydrate | | |
| Amoxapine | Demecarium bromide | Rifapentine |
| Cyproheptadine hydrochloride | Quipazine dimaleate salt | Ropinirole HCl |
| Famotidine | Diflorasone Diacetate | Sibutramine HCl |
| Danazol | Harmane hydrochloride | Stanozolol |
| Nicorandil | Methoxy-6-harmalan | Zonisamide |
| Nomifensine maleate | Pyridoxine hydrochloride | Acitretin |
| Dizocilpine maleate | Racecadotril | Rebamipide |
| Naloxone hydrochloride | Folic acid | Diacerein |
| Metolazone | Dimethisoquin hydrochloride | Miglitol |
| Ciprofloxacin hydrochloride | Dipivefrin hydrochloride | Venlafaxine |
| Ampicillin trihydrate | Thiorphan | Irsogladine Maleate |
| Haloperidol | Sulmazole | Acarbose |
| Naltrexone hydrochloride dihydrate | Flunisolide | Carbidopa |
| Chlorpheniramine maleate | N-Acetyl-DL-homocysteine | Aniracetam |
| | Thiolactone | |
| Nalbuphine hydrochloride | Flurandrenolide | Busulfan |
| Picotamide monohydrate | Etanidazole | Docetaxel |
| Triamcinolone | Butirosin disulfate salt | Tibolone |
| Bromocryptine mesylate | Glimepiride | Tizanidine HCl |
| Dehydrocholic acid | Picrotoxinin | Temozolomide |
| Perphenazine | Mepenzolate bromide | Tioconazole |
| Mefloquine hydrochloride | Benfotiamine | granisetron |
| Isoconazole | Halcinonide | ziprasidone Hydrochloride |
| Spironolactone | Lanatoside C | montelukast |
| Pirenzepine dihydrochloride | Benzamil hydrochloride | olmesartan |
| Dexamethasone acetate | Suxibuzone | Oxandrolone |
| Glipizide | 6-Furfurylaminopurine | Thimerosal |
| Loxapine succinate | Avennectin B1a | toltrazuril |
| Hydroxyzine dihydrochloride | Nisoldipine | topotecan |
| Diltiazem hydrochloride | Foliosidine | Toremifene |
| Methotrexate | Dydrogesterone | tranilast |
| Astemizole | Beta-Escin | Tripelennamine hydrochloride |
| Clindamycin hydrochloride | Pempidine tartrate | Clindamycin Phosphate |
| Terfenadine | Nitrarine dihydrochloride | 4-aminosalicylic acid |
| Cefotaxime sodium salt | Estropipate | 5-fluorouracil |
| Tetracycline hydrochloride | Citalopram Hydrobromide | acetylcysteine |
| Verapamil hydrochloride | Promazine hydrochloride | acetylsalicylic acid |
| Dipyridamole | Sulfamerazine | alendronate sodium |
| Chlorhexidine | Ethotoin | alfacalcidol |
| Loperamide hydrochloride | 3-alpha-Hydroxy-5-beta-androstan- | Allopurinol |
| | 17-one | |
| Chlortetracycline hydrochloride | Tetrahydrozoline hydrochloride | amisulpride |
| Tamoxifen citrate | Hexestrol | Amlodipine |
| Nicergoline | Cefmetazole sodium salt | anastrozole |
| Canrenoic acid potassium salt | Trihexyphenidyl-D,L | anethole-trithione |
| | Hydrochloride | |
| Thioproperazine dimesylate | Succinylsulfathiazole | Anthralin |
| Dihydroergotamine tartrate | Famprofazone | argatroban |
| Erythromycin | Bromopride | aripiprazole |
| Didanosine | Methyl benzethonium chloride | atorvastatin |
| Josamycin | Chlorcyclizine hydrochloride | auranofin |
| Paclitaxel | Diphenylpyraline hydrochloride | Azithromycin |
| Ivermectin | Benzethonium chloride | Benztropine mesylate |
| Gallamine triethiodide | Trioxsalen | bicalutamide |
| Neomycin sulfate | Sulfabenzamide | bifonazole |
| Dihydrostreptomycin sulfate | Benzocaine | erlotinib |
| Gentamicin sulfate | Dipyrone | bosentan |
| Isoniazid | Isosorbide dinitrate | bromhexine |
| Pentylenetetrazole | Sulfachloropyridazine | famciclovir |
| Chlorzoxazone | Pramoxine hydrochloride | Butalbital |
| Ornidazole | Finasteride | butenafine |
| Ethosuximide | Fluorometholone | butylscopolammonium (n-) |
| | | bromide |
| Mafenide hydrochloride | Cephalothin sodium salt | fentiazac |
| Riluzole hydrochloride | Cefuroxime sodium salt | caffeine |
| Nitrofurantoin | Althiazide | calcipotriene |
| Hydralazine hydrochloride | Isopyrin hydrochloride | candesartan |
| Phenelzine sulfate | Phenethicillin potassium salt | canrenone |
| Tranexamic acid | Sulfamethoxypyridazine | carprofen |
| Etofylline | Deferoxamine mesylate | carvedilol |
| Tranylcypromine hydrochloride | Mephentermine hemisulfate | Cefdinir |
| Alverine citrate salt | Sulfadimethoxine | gatifloxacin |
| Aceclofenac | Sulfanilamide | gemcitabine |
| Iproniazide phosphate | Balsalazide Sodium | gestrinone |
| Sulfamethoxazole | Sulfaquinoxaline sodium salt | guaiacol |
| Mephenesin | Streptozotocin | gefitinib |
| Phenformin hydrochloride | Metoprolol-(+,−) (+)-tartrate salt | Escitalopram |
| Flutamide | Flumethasone | emedastine |
| Ampyrone | Flecainide acetate | Stavudine |
| Levamisole hydrochloride | Cefazolin sodium salt | mepivacaine hydrochloride |
| Pargyline hydrochloride | Atractyloside potassium salt | Methenamine |
| Methocarbamol | Folinic acid calcium salt | Buspirone hydrochloride |
| Aztreonam | Levonordefrin | ibandronate |
| Cloxacillin sodium salt | Ebselen | ibudilast |
| Catharanthine | Nadide | idebenone |
| Pentolinium bitartrate | Sulfamethizole | imatinib |
| Aminopurine, 6-benzyl | Medrysone | imiquimod |
| Tolbutamide | Flunixin meglumine | ipsapirone |
| Midodrine hydrochloride | Spiramycin | Isosorbide mononitrate |
| Thalidomide | Glycopyrrolate | itopride |
| Oxolinic acid | Cefamandole sodium salt | lacidipine |
| Nimesulide | Monensin sodium salt | lamivudine |
| Hydrastinine hydrochloride | Isoetharine mesylate salt | lapatinib ditosylate |
| Pentoxifylline | Mevalonic-D,L acid lactone | pefloxacine |
| Metaraminol bitartrate | Terazosin hydrochloride | olopatadine |
| Salbutamol | Phenazopyridine hydrochloride | phentermine hydrochloride |
| Prilocaine hydrochloride | Demeclocycline hydrochloride | Phenylbutazone |
| Camptothecine (S,+) | Fenoprofen calcium salt dihydrate | pioglitazone |
| Ranitidine hydrochloride | Piperacillin sodium salt | potassium clavulanate |
| Tiratricol, 3,3′,5-triiodothyroacetic | Diethylstilbestrol | pramipexole |
| acid | | |
| Flufenamic acid | Chlorotrianisene | pranlukast |
| Flumequine | Ribostarmycin sulfate salt | Pranoprofen |
| Tolfenamic acid | Methacholine chloride | Pravastatin |
| Meclofenamic acid sodium salt | Pipenzolate bromide | Prothionamide |
| monohydrate | | |
| Trimethoprim | Butamben | Pyridostigmine iodid |
| Metoclopramide monohydrochloride | Sulfapyridine | Quetiapine |
| Fenbendazole | Meclofenoxate hydrochloride | raclopride |
| Piroxicam | Furaltadone hydrochloride | reboxetine mesylate |
| Pyrantel tartrate | Ethoxyquin | Rimantadine |
| Fenspiride hydrochloride | Tinidazole | rivastigmine |
| Gemfibrozil | Guanadrel sulfate | rofecoxib |
| Mefexamide hydrochloride | Vidarabine | rosiglitazone |
| Tiapride hydrochloride | Sulfameter | rufloxacin |
| Mebendazole | Isopropamide iodide | sarafloxacin |
| Fenbufen | Alclometasone dipropionate | secnidazole |
| Ketoprofen | Leflunomide | sertindole |
| Indapamide | Norgestrel-(−)-D | sildenafil |
| Norfloxacin | Fluocinonide | sparfloxacin |
| Antimycin A | Sulfamethazine sodium salt | sulbactam |
| Xylometazoline hydrochloride | Guaifenesin | sumatriptan succinate |
| Oxymetazoline hydrochloride | Alexidine dihydrochloride | tazobactam |
| Nifenazone | Proadifen hydrochloride | telmisartan |
| Griseofulvin | Zomepirac sodium salt | tenatoprazole |
| Clemizole hydrochloride | Cinoxacin | tulobuterol |
| Tropicamide | Clobetasol propionate | tylosin |
| Nefopam hydrochloride | Podophyllotoxin | vardenafil |
| Phentolamine hydrochloride | Clofibric acid | vatalanib |
| Etodolac | Bendroflumethiazide | vecuronium bromide |
| Scopolamin-N-oxide hydrobromide | Dicumarol | Viloxazine hydrochloride |
| Hyoscyamine (L) | Methimazole | vorinostat |
| Chlorphensin carbamate | Merbromin | Warfarin |
| Metampicillin sodium salt | Hexylcaine hydrochloride | zafirlukast |
| Dilazep dihydrochloride | Drofenine hydrochloride | zileuton |
| Ofloxacin | Cycloheximide | zopiclone |
| Lomefloxacin hydrochloride | (R)-Naproxen sodium salt | zotepine |
| Orphenadrine hydrochloride | Propidium iodide | zaleplon |
| Proglumide | Cloperastine hydrochloride | celecoxib |
| Mexiletine hydrochloride | Eucatropine hydrochloride | chlormadinone acetate |
| Flavoxate hydrochloride | Isocarboxazid | cilnidipine |
| Bufexamac | Lithocholic acid | Clarithromycin |
| Glutethimide, para-amino | Methotrimeprazine maleat salt | clobutinol hydrochloride |
| Dropropizine (R,S) | Dienestrol | clodronate |
| Pinacidil | Pridinol methanesulfonate salt | clofibrate |
| Albendazole | Amrinone | closantel |
| Clonidine hydrochloride | Carbinoxamine maleate salt | desloratadine |
| Bupropion hydrochloride | Methazolamide | Dexfenfluramine hydrochloride |
| Alprenolol hydrochloride | Pyrithyldione | Dibenzepine hydrochloride |
| Chlorothiazide | Spectinomycin dihydrochloride | diclazuril |
| Diphenidol hydrochloride | Piromidic acid | dopamine hydrochloride |
| Norethindrone | Trimipramine maleate salt | doxycycline hydrochloride |
| Nortriptyline hydrochloride | Chloropyramine hydrochloride | Efavirenz |
| Niflumic acid | Furazolidone | Enoxacin |
| Isotretinoin | Dichlorphenamide | Entacapone |
| Retinoic acid | Sulconazole nitrate | Ethinylestradiol |
| Antazoline hydrochloride | Cromolyn disodium salt | Etofenamate |
| Ethacrynic acid | Bucladesine sodium salt | Etoricoxib |
| Praziquantel | Cefsulodin sodium salt | Etretinate |
| Ethisterone | Fosfosal | Exemestane |
| Triprolidine hydrochloride | Suprofen | fleroxacin |
| Doxepin hydrochloride | Catechin-(+,−) hydrate | floxuridine |
| Dyclonine hydrochloride | Nadolol | flubendazol |
| Dimenhydrinate | Moxalactam disodium salt | Fluconazole |
| Disopyramide | Aminophylline | fluocinolone acetonide |
| Clotrimazole | Azlocillin sodium salt | formestane |
| Vinpocetine | Clidinium bromide | formoterol fumarate |
| Clomipramine hydrochloride | Sulfamonomethoxine | Fosinopril |
| Fendiline hydrochloride | Benzthiazide | fulvestrant |
| Vincamine | Trichlormethiazide | levetiracetam |
| Indomethacin | Oxalamine citrate salt | linezolid |
| Cortisone | Propantheline bromide | lofexidine |
| Prednisolone | Dimethadione | loratadine |
| Fenofibrate | Ethaverine hydrochloride | losartan |
| Bumetanide | Butacaine | melengestrol acetate |
| Labetalol hydrochloride | Cefoxitin sodium salt | mevastatin |
| Cinnarizine | Ifosfamide | Misoprostol |
| Methylprednisolone, 6-alpha | Novobiocin sodium salt | Mitotane |
| Quinidine hydrochloride | Tetrahydroxy-1,4-quinone | moxifloxacin |
| monohydrate | monohydrate | |
| Fludrocortisone acetate | Indoprofen | Nalidixic acid sodium salt |
| Fenoterol hydrobromide | Carbenoxolone disodium salt | nicotinamide |
| Homochlorcyclizine dihydrochloride | Iocetamic acid | Norgestimate |
| Diethylcarbamazine citrate | Ganciclovir | Nylidrin |
| Chenodiol | Ethopropazine hydrochloride | olanzapine |
| Perhexiline maleate | Trimeprazine tartrate | opipramol dihydrochloride |
| Oxybutynin chloride | Nafcillin sodium salt monohydrate | oxfendazol |
| Spiperone | Procyclidine hydrochloride | oxibendazol |
| Pyrilamine maleate | Amiprilose hydrochloride | tomoxetine hydrochloride |
| Sulfinpyrazone | Ethynylestradiol 3-methyl ether | Tosufloxacin hydrochloride |
| Dantrolene sodium salt | (−)-Levobunolol hydrochloride | Tramadol hydrochloride |
| Trazodone hydrochloride | Iodixanol | troglitazone |
| Glafenine hydrochloride | Rolitetracycline | Mercaptopurine |
| Pimethixene maleate | Equilin | Amfepramone hydrochloride |
| Pergolide mesylate | Paroxetine Hydrochloride | Hexachlorophene |
| Acemetacin | Liothyronine | Estradiol Valerate |
| Benzydamine hydrochloride | Roxithromycin | Chloroxine |
| Fipexide hydrochloride | Beclomethasone dipropionate | Oxacillin Na |
| Mifepristone | Tolmetin sodium salt dihydrate | Amcinonide |
| Diperodon hydrochloride | (+)-Levobunolol hydrochloride | Penicillamine |
| Lisinopril | Doxazosin mesylate | Rifaximin |
| Lincomycin hydrochloride | Fluvastatin sodium salt | Triclosan |
| Telenzepine dihydrochloride | Methylhydantoin-5-(L) | Racepinephrine HCl |
| Econazole nitrate | Gabapentin | cyclophosphamide |
| Bupivacaine hydrochloride | Raloxifene hydrochloride | Valproic acid |
| Clemastine fumarate | Etidronic acid. disodium salt | Fludarabine |
| Oxytetracycline dihydrate | Methylhydantoin-5-(D) | Cladribine |
| Pimozide | Simvastatin | Cortisol acetate |
| Amodiaquin dihydrochloride | Azacytidine-5 | Mesna |
| dihydrate | | |
| Mebeverine hydrochloride | Paromomycin sulfate | Penciclovir |
| Ifenprodil tartrate | Acetaminophen | amifostine |
| Flunarizine dihydrochloride | Phthalylsulfathiazole | Nalmefene |
| Trifluoperazine dihydrochloride | Luteolin | Pentobarbital |
| Enalapril maleate | Iopamidol | Lamotrigine |
| Minocycline hydrochloride | Iopromide | Topiramate |
| Glibenclamide | Theophylline monohydrate | Irinotecan Hydrochloride |
| Guanethidine sulfate | Theobromine | Rabeprazole |
| Quinacrine dihydrochloride dihydrate | Reserpine | Tiludronate disodium |
| Clofilium tosylate | Scopolamine hydrochloride | Ambrisentan |
| Fluphenazine dihydrochloride | Ioversol | Torsemide |
| Streptomycin sulfate | Carbachol | Halofantrine hydrochloride |
| Alfuzosin hydrochloride | Niacin | Articaine hydrochloride |
| Chlorpropamide | Bemegride | Nomegestrol acetate |
| Phenylpropanolamine hydrochloride | Digoxigenin | Pancuronium bromide |
| Ascorbic acid | Meglumine | Molindone hydrochloride |
| Methyldopa (L,−) | Cantharidin | Alcuronium chloride |
| Cefoperazone dihydrate | Clioquinol | Zalcitabine |
| Zoxazolamine | Oxybenzone | Methyldopate hydrochloride |
| Tacrine hydrochloride hydrate | Promethazine hydrochloride | Levocabastine hydrochloride |
| Bisoprolol fumarate | FeIbinac | Pyrvinium pamoate |
| Tremorine dihydrochloride | Butylparaben | Etomidate |
| Practolol | Aminohippuric acid | Tridihexethyl chloride |
| Zidovudine, AZT | N-Acetyl-L-leucine | Penbutolol sulfate |
| Sulfisoxazole | Pipemidic acid | Prednicarbate |
| Zaprinast | Dioxybenzone | Sertaconazole nitrate |
| Chlormezanone | Adrenosterone | Repaglinide |
| Procainamide hydrochloride | Methylatropine nitrate | Piretanide |
| N6-methyladenosine | Hymecromone | Piperacetazine |
| Guanfacine hydrochloride | Caffeic acid | Oxyphenbutazone |
| Domperidone | Diloxanide furoate | Quinethazone |
| Furosemide | Metyrapone | Moricizine hydrochloride |
| Methapyrilene hydrochloride | Urapidil hydrochloride | Iopanoic acid |
| Desipramine hydrochloride | Fluspirilen | Pivmecillinam hydrochloride |
| Clorgyline hydrochloride | S-(+)-ibuprofen | Levopropoxyphene napsylate |
| Clenbuterol hydrochloride | Ethynodiol diacetate | Piperidolate hydrochloride |
| Maprotiline hydrochloride | Nabumetone | Trifluridine |
| Thioguanosine | Nisoxetine hydrochloride | Oxprenolol hydrochloride |
| Chlorprothixene hydrochloride | (+)-Isoproterenol (+)-bitartrate salt | Ondansetron Hydrochloride |
| Ritodrine hydrochloride | Monobenzone | Propoxycaine hydrochloride |
| Clozapine | 2-Aminobenzenesulfonamide | Oxaprozin |
| Chlorthalidone | Estrone | Phensuximide |
| Dobutamine hydrochloride | Lorglumide sodium salt | Ioxaglic acid |
| Moclobemide | Nitrendipine | Naftifine hydrochloride |
| Clopamide | Flurbiprofen | Meprylcaine hydrochloride |
| Hycanthone | Nimodipine | Milrinone |
| Adenosine 5′-monophosphate | Bacitracin | Methantheline bromide |
| monohydrate | | |
| Amoxicillin | L(−)-vesamicol hydrochloride | Ticarcillin sodium |
| Cephalexin monohydrate | Nizatidine | Thiethylperazine malate |
| Dextromethorphan hydrobromide | Thioperamide maleate | Mesalamine |
| monohydrate | | |
| Droperidol | Xamoterol hemifumarate | Imidurea |
| Bambuterol hydrochloride | Rolipram | Lansoprazole |
| Betamethasone | Thonzonium bromide | Bethanechol chloride |
| Colchicine | Idazoxan hydrochloride | Cyproterone acetate |
| Metergoline | Quinapril HCl | (R)-Propranolol hydrochloride |
| Brinzolamide | Nilutamide | Ciprofibrate |
| Ambroxol hydrochloride | Ketorolac tromethamine | Benzylpenicillin sodium |
| Benfluorex hydrochloride | Protriptyline hydrochloride | Chlorambucil |
| Bepridil hydrochloride | Propofol | Methiazole |
| Meloxicam | S(−)Eticlopride hydrochloride | (S)-propranolol hydrochloride |
| Benzbromarone | Primidone | (−)-Eseroline fumarate salt |
| Ketotifen fumarate | Flucytosine | Leucomisine |
| Debrisoquin sulfate | (−)-MK 801 hydrogen maleate | D-cycloserine |
| Amethopterin (R,S) | Bephenium hydroxynaphthoate | 2-Chloropyrazine |
| Methylergometrine maleate | Dehydroisoandosterone 3-acetate | (+,−)-Synephrine |
| Methiothepin maleate | Benserazide hydrochloride | (S)-(−)-Cycloserine |
| Clofazimine | Iodipamide | Homosalate |
| Nafronyl oxalate | Pentetic acid | Spaglumic acid |
| Bezafibrate | Bretylium tosylate | Ranolazine |
| Clebopride maleate | Pralidoxime chloride | Sulfadoxine |
| Lidoflazine | Phenoxybenzamine hydrochloride | Cyclopentolate hydrochloride |
| Betaxolol hydrochloride | Salmeterol | Estriol |
| Nicardipine hydrochloride | Altretamine | (−)-Isoproterenol hydrochloride |
| Probucol | Prazosin hydrochloride | Nialamide |
| Mitoxantrone dihydrochloride | Timolol maleate salt | Perindopril |
| GBR 12909 dihydrochloride | (+,−)-Octopamine hydrochloride | Fexofenadine HCl |
| Carbetapentane citrate | Crotamiton | Clonixin Lysinate |
| Dequalinium dichloride | (S)-(−)-Atenolol | Verteporfin |
| Ketoconazole | Tyloxapol | Meropenem |
| Fusidic acid sodium salt | Florfenicol | Ramipril |
| Terbutaline hemisulfate | Megestrol acetate | Mephenytoin |
| Ketanserin tartrate hydrate | Deoxycorticosterone | Rifabutin |
| Hemicholinium bromide | Urosiol | Parbendazole |
| Kanamycin A sulfate | Proparacaine hydrochloride | Mecamylamine hydrochloride |
| Amikacin hydrate | Aminocaproic acid | Procarbazine hydrochloride |
| Etoposide | Denatonium benzoate | Viomycin sulfate |
| Clomiphene citrate (Z,E) | Enilconazole | Saquinavir mesylate |
| Oxantel pamoate | Methacycline hydrochloride | Ronidazole |
| Prochlorperazine dimaleate | Sotalol hydrochloride | Dorzolamide hydrochloride |
| Hesperidin | Decamethonium bromide | Azaperone |
| Testosterone propionate | 3-Acetamidocoumarin | Cefepime hydrochloride |
| Arecoline hydrobromide | Roxarsone | Clocortolone pivalate |
| Thyroxine (L) | Remoxipride Hydrochloride | Nadifloxacin |
| Pepstatin A | THIP Hydrochloride | Carbadox |
| SR-95639A | Pirlindole mesylate | Oxiconazole Nitrate |
| Adamantamine fumarate | Pronethalol hydrochloride | Acipimox |
| Butoconazole nitrate | Naftopidil dihydrochloride | Benazepril HCl |
| Amiodarone hydrochloride | Tracazolate hydrochloride | Azelastine HCl |
| Amphotericin B | Zardaverine | Celiprolol HCl |
| Androsterone | Memantine Hydrochloride | Cytarabine |
| Carbarsone | Ozagrel hydrochloride | Doxofylline |
| Bacampicillin hydrochloride | Piribedil hydrochloride | Esmolol hydrochloride |
| Biotin | Nitrocaramiphen hydrochloride | Itraconazole |
| Bisacodyl | Nandrolone | Liranaftate |
| Suloctidil | Dimaprit dihydrochloride | Mirtazapine |
| Carisoprodol | Proscillaridin A | Modafinil |
| Cephalosporanic acid, 7-amino | Gliquidone | Nefazodone HCl |
| Chicago sky blue 6B | Pizotifen malate | Nilvadipine |
| Buflomedil hydrochloride | Ribavirin | Oxcarbazepine |
| Roxatidine Acetate HCl | Cyclopenthiazide | Rifapentine |
| Cholecalciferol | Fluvoxamine maleate | Ropinirole HCl |
| Cisapride | Fluticasone propionate | Sibutramine HCl |
| Corticosterone | Zuclopenthixol hydrochloride | Stanozolol |
| Cyanocobalamin | Proguanil hydrochloride | Zonisamide |
| Cefadroxil | Lymecycline | Acitretin |
| Cyclosporin A | Alfadolone acetate | Rebamipide |
| Digitoxigenin | Alfaxalone | Diacerein |
| Digoxin | Azapropazone | Miglitol |
| Doxorubicin hydrochloride | Meptazinol hydrochloride | Venlafaxine |
| Carbimazole | Apramycin | Irsogladine Maleate |
| Epiandrosterone | Epitiostanol | Acarbose |
| Estradiol-17 beta | Fursultiamine Hydrochloride | Carbidopa |
| Gabazine | Gabexate mesilate | Aniracetam |
| Cyclobenzaprine hydrochloride | Pivampicillin | Busulfan |
| Carteolol hydrochloride | Talampicillin hydrochloride | Docetaxel |
| Hydrocortisone base | Flucloxacillin sodium | Tibolone |
| Hydroxytacrine maleate (R,S) | Trapidil | Tizanidine HCl |
| Pilocarpine nitrate | Deptropine citrate | Temozolomide |
| Dicloxacillin sodium salt | Sertraline | Tioconazole |
| Alizapride HCl | Ethamsylate | granisetron |
| Mebhydroline 1,5- | Moxonidine | ziprasidone Hydrochloride |
| naphtalenedisulfonate | | |
| Meclocycline sulfosalicylate | Etilefrine hydrochloride | montelukast |
| Meclozine dihydrochloride | Alprostadil | olmesartan |
| Melatonin | Tribenoside | Oxandrolone |
| Dinoprost trometamol | Rimexolone | Thimerosal |
| Tropisetron HCl | Isradipine | toltrazuril |
| Cefixime | Tiletamine hydrochloride | topotecan |
| Metrizamide | Isometheptene mucate | Toremifene |
| Neostigmine bromide | Nifurtimox | tranilast |
| Niridazole | Letrozole | Tripelennamine hydrochloride |
| Ceforanide | Arbutin | Clindamycin Phosphate |
| Cefotetan | Tocainide hydrochloride | 4-aminosalicylic acid |
| Brompheniramine maleate | Benzathine benzylpenicillin | 5-fluorouracil |
| Azaguanine-8 | Risperidone | acetylcysteine |

TABLE 5

| | | |
|---|---|---|
| MAFENIDE HYDROCHLORIDE | CYPROTERONE ACETATE | BENDROFLUMETHIAZIDE |
| MAPROTILINE | CYTARABINE | BEPRIDIL HYDROCHLORIDE |
| HYDROCHLORIDE | | |
| MECAMYLAMINE | DACARBAZINE | BROMHEXINE |
| HYDROCHLORIDE | | HYDROCHLORIDE |
| MECHLORETHAMINE | DANAZOL | CARMUSTINE |
| MECLIZINE HYDROCHLORIDE | DAPSONE | CEFTRIAXONE SODIUM |
| | | TRIHYDRATE |
| MECLOFENAMATE SODIUM | DAUNORUBICIN | TRIMIPRAMINE MALEATE |
| MEDRYSONE | SODIUM DEHYDROCHOLATE | TRIFLUPROMAZINE |
| | | HYDROCHLORIDE |
| MEGESTROL ACETATE | DEMECLOCYCLINE | TRAZODONE |
| | HYDROCHLORIDE | HYDROCHLORIDE |
| MELPHALAN | DESIPRAMINE | MENTHOL(−) |
| | HYDROCHLORIDE | |
| MESTRANOL | DEXAMETHASONE | THONZYLAMINE |
| | | HYDROCHLORIDE |
| METAPROTERENOL | DEXAMETHASONE ACETATE | THIAMPHENICOL |
| METHACHOLINE CHLORIDE | DEFEROXAMINE MESYLATE | TENOXICAM |
| METHIMAZOLE | DEXAMETHASONE SODIUM | CHLOROXINE |
| | PHOSPHATE | |
| METHOCARBAMOL | DEXTROMETHORPHAN | CHLORPROTHIXENE |
| | HYDROBROMIDE | HYDROCHLORIDE |
| METHOTREXATE(+/−) | DIBENZOTHIOPHENE | CINNARAZINE |
| METHOXAMINE | DIBUCAINE | DANTROLENE SODIUM |
| HYDROCHLORIDE | HYDROCHLORIDE | |
| METHYLDOPA | DICLOFENAC SODIUM | BETAMETHASONE 17,21- |
| | | DIPROPIONATE |
| METHYLPREDNISOLONE | DICLOXACILLIN SODIUM | DOBUTAMINE |
| | | HYDROCHLORIDE |
| METOCLOPRAMIDE | DICUMAROL | EDOXUDINE |
| HYDROCHLORIDE | | |
| METOPROLOL TARTRATE | DICYCLOMINE | ENOXACIN |
| | HYDROCHLORIDE | |
| METRONIDAZOLE | DIENESTROL | ETHISTERONE |
| MINOCYCLINE | DIETHYLCARBAMAZINE | PARAROSANILINE PAMOATE |
| HYDROCHLORIDE | CITRATE | |
| MINOXIDIL | DIETHYLSTILBESTROL | PERHEXILINE MALEATE |
| MOXALACTAM DISODIUM | DIFLUNISAL | PAROMOMYCIN SULFATE |
| NADIDE | DIGITOXIN | METHAPYRILENE |
| | | HYDROCHLORIDE |
| NAFCILLIN SODIUM | DIGOXIN | BETA-PROPIOLACTONE |
| NALOXONE HYDROCHLORIDE | DIHYDROERGOTAMINE | HALCINONIDE |
| | MESYLATE | |
| NAPHAZOLINE | DIHYDROSTREPTOMYCIN | HYCANTHONE |
| HYDROCHLORIDE | SULFATE | |
| NAPROXEN(+) | DIMENHYDRINATE | PYRIDOSTIGMINE BROMIDE |
| NEOSTIGMINE BROMIDE | DIMETHADIONE | ISOXICAM |
| NIACIN | DIOXYBENZONE | LABETALOL |
| | | HYDROCHLORIDE |
| NIFEDIPINE | DIPHENHYDRAMINE | LEVAMISOLE |
| | HYDROCHLORIDE | HYDROCHLORIDE |
| NITROFURANTOIN | DIPHENYLPYRALINE | MEPHENTERMINE SULFATE |
| | HYDROCHLORIDE | |
| OXYBUTYNIN CHLORIDE | DIPYRIDAMOLE | METARAMINOL BITARTRATE |
| NOREPINEPHRINE | PYRITHIONE ZINC | METHAZOLAMIDE |
| NORETHINDRONE | DISOPYRAMIDE PHOSPHATE | METHYLBENZETHONIUM |
| | | CHLORIDE |
| NORETHYNODREL | DISULFIRAM | METHYLPREDNISOLONE |
| | | SODIUM SUCCINATE |
| NORFLOXACIN | DOPAMINE | AMSACRINE |
| | HYDROCHLORIDE | |
| NORGESTREL | DOXEPIN HYDROCHLORIDE | MIDODRINE |
| | | HYDROCHLORIDE |
| NORTRIPTYLINE | DOXYCYCLINE | NADOLOL |
| | HYDROCHLORIDE | |
| NOSCAPINE HYDROCHLORIDE | DOXYLAMINE SUCCINATE | NALTREXONE |
| | | HYDROCHLORIDE |
| NOVOBIOCIN SODIUM | DYCLONINE | CYCLOTHIAZIDE |
| | HYDROCHLORIDE | |
| NYLIDRIN HYDROCHLORIDE | DYPHYLLINE | NICLOSAMIDE |
| NYSTATIN | TRISODIUM | NOMIFENSINE MALEATE |
| | ETHYLENEDIAMINE | |
| | TETRACETATE | |
| ORPHENADRINE CITRATE | EMETINE | PERGOLIDE MESYLATE |
| OXACILLIN SODIUM | ADRENALINE BITARTRATE | PRILOCAINE |
| | | HYDROCHLORIDE |
| OXYBENZONE | EQUILIN | HYDROCORTISONE |
| | | BUTYRATE |
| OXYMETAZOLINE | ERGOCALCIFEROL | ROXITHROMYCIN |
| HYDROCHLORIDE | | |
| OXYPHENBUTAZONE | ERGONOVINE MALEATE | MITOXANTHRONE |
| | | HYDROCHLORIDE |
| OXYTETRACYCLINE | ERYTHROMYCIN | OXETHAZAINE |
| | ETHYLSUCCINATE | |
| PAPAVERINE | ESTRADIOL | DIPYRONE |
| HYDROCHLORIDE | | |
| PARACHLOROPHENOL | ESTRADIOL CYPIONATE | SULFANILATE ZINC |
| PARGYLINE HYDROCHLORIDE | ESTRADIOL VALERATE | URETHANE |
| PENICILLAMINE | ESTRIOL | THIRAM |
| PHENACEMIDE | ESTRONE | THIOTEPA |
| PHENAZOPYRIDINE | ETHACRYNIC ACID | TETROQUINONE |
| HYDROCHLORIDE | | |
| PHENELZINE SULFATE | ETHAMBUTOL | SULFANITRAN |
| | HYDROCHLORIDE | |
| PHENINDIONE | ETHINYL ESTRADIOL | OXIBENDAZOLE |
| PHENIRAMINE MALEATE | ETHIONAMIDE | PIPOBROMAN |
| PHENYLBUTAZONE | ETHOPROPAZINE | ETANIDAZOLE |
| | HYDROCHLORIDE | |
| PHENYTOIN SODIUM | EUCATROPINE | NAFRONYL OXALATE |
| | HYDROCHLORIDE | |
| FENOFIBRATE | EUGENOL | QUIPAZINE MALEATE |
| FENOPROFEN | FLUDROCORTISONE | RITANSERIN |
| | ACETATE | |
| FLUFENAMIC ACID | FLUMETHAZONE PIVALATE | SEMUSTINE |
| FENBENDAZOLE | FLUOCINOLONE ACETONIDE | SPIRAMYCIN |
| FENSPIRIDE HYDROCHLORIDE | FLUOCINONIDE | CLOFIBRATE |
| MEFENAMIC ACID | FLUOROMETHOLONE | RESORCINOL MONOACETATE |
| METHACYCLINE | FLUOROURACIL | NIMODIPINE |
| HYDROCHLORIDE | | |
| MEFEXAMIDE | FLURBIPROFEN | ACYCLOVIR |
| PROBUCOL | FURAZOLIDONE | RETINYL PALMITATE |
| PUROMYCIN | FUROSEMIDE | THALIDOMIDE |
| HYDROCHLORIDE | | |
| MEBENDAZOLE | FUSIDIC ACID | NITRENDIPINE |
| NALBUPHINE | GALLAMINE TRIETHIODIDE | BENZALKONIUM CHLORIDE |
| HYDROCHLORIDE | | |
| PROGLUMIDE | GEMFIBROZIL | CIPROFLOXACIN |
| MINAPRINE HYDROCHLORIDE | GENTAMICIN SULFATE | CELECOXIB |
| MEMANTINE | GENTIAN VIOLET | AZITHROMYCIN |
| HYDROCHLORIDE | | |
| ATENOLOL | GLUCOSAMINE | ANETHOLE |
| | HYDROCHLORIDE | |
| CARBETAPENTANE CITRATE | GRAMICIDIN | TERFENADINE |
| PIMOZIDE | GUAIFENESIN | CLOPIDOGREL SULFATE |
| NICARDIPINE | GUANABENZ ACETATE | LORATADINE |
| HYDROCHLORIDE | | |
| NEFOPAM | GUANETHIDINE SULFATE | SELAMECTIN |
| PIRENZEPINE | HALAZONE | NAPROXOL |
| HYDROCHLORIDE | | |
| PRAMOXINE | HALOPERIDOL | COLFORSIN |
| HYDROCHLORIDE | | |
| MEPHENESIN | HETACILLIN POTASSIUM | ISOSORBIDE MONONITRATE |
| SULFACHLORPYRIDAZINE | HEXACHLOROPHENE | AMCINONIDE |
| SULFADIMETHOXINE | HEXYLRESORCINOL | BUPIVACAINE |
| | | HYDROCHLORIDE |
| SULFAGUANIDINE | HISTAMINE | ALBENDAZOLE |
| | DIHYDROCHLORIDE | |
| SULFAMONOMETHOXINE | HOMATROPINE BROMIDE | PACLITAXEL |
| SULCONAZOLE NITRATE | HOMATROPINE | BUTACAINE |
| | METHYLBROMIDE | |
| RITODRINE HYDROCHLORIDE | HYDRALAZINE | CLOBETASOL PROPIONATE |
| | HYDROCHLORIDE | |
| SULPIRIDE | HYDROCHLOROTHIAZIDE | IOPANIC ACID |
| RANITIDINE | HYDROCORTISONE ACETATE | KETOROLAC |
| | | TROMETHAMINE |
| SULOCTIDIL | HYDROCORTISONE | LANSOPRAZOLE |
| | HEMISUCCINATE | |
| RONIDAZOLE | HYDROCORTISONE | MEXILETINE |
| | PHOSPHATE | HYDROCHLORIDE |
| | TRIETHYLAMINE | |
| SULFAMETER | HYDROFLUMETHIAZIDE | MORANTEL CITRATE |
| SULFAMETHOXYPYRIDAZINE | HYDROXYPROGESTERONE | PERPHENAZINE |
| | CAPROATE | |
| SUPROFEN | HYDROXYUREA | RIBAVIRIN |
| SACCHARIN | HYDROXYZINE PAMOATE | TACROLIMUS |
| ACETANILIDE | HYOSCYAMINE | BROMPHENIRAMINE |
| | | MALEATE |
| FLURANDRENOLIDE | IBUPROFEN | SIROLIMUS |
| ESTRADIOL ACETATE | IMIPRAMINE | PAROXETINE |
| | HYDROCHLORIDE | HYDROCHLORIDE |
| ECONAZOLE NITRATE | INDAPAMIDE | ETHYLNOREPINEPHRINE |
| | | HYDROCHLORIDE |
| FLUNISOLIDE | INDOMETHACIN | ALAPROCLATE |
| FLUMETHASONE | INDOPROFEN | ACETRIAZOIC ACID |
| XYLAZINE | INOSITOL | VENLAFAXINE |
| TOLAZAMIDE | IODOQUINOL | CITALOPRAM |
| GALANTHAMINE | IPRATROPIUM BROMIDE | FLUOXETINE |
| HYDROBROMIDE | | |
| LANATOSIDE C | ISONIAZID | BUPROPION |
| ENALAPRIL MALEATE | ISOPROPAMIDE IODIDE | CEFUROXIME AXETIL |
| KETOPROFEN | ISOPROTERENOL | FEXOFENADINE |
| | HYDROCHLORIDE | HYDROCHLORIDE |
| LISINOPRIL | ISOSORBIDE DINITRATE | TRIFLURIDINE |
| BUMETANIDE | ISOXSUPRINE | PIRENPERONE |
| | HYDROCHLORIDE | |
| CARBENOXOLONE SODIUM | KANAMYCIN A SULFATE | AVOBENZONE |
| FOLIC ACID | KETOCONAZOLE | ATOVAQUONE |
| PHTHALYLSULFATHIAZOLE | LACTULOSE | TRIMETOZINE |
| SUCCINYLSULFATHIAZOLE | LEUCOVORIN CALCIUM | ZOXAZOLAMINE |
| TRANEXAMIC ACID | LEVONORDEFRIN | CYSTEAMINE |
| | | HYDROCHLORIDE |
| CEPHALEXIN | LINCOMYCIN | ROFECOXIB |
| | HYDROCHLORIDE | |
| OXOLINIC ACID | MEDROXYPROGESTERONE | SIMVASTATIN |
| | ACETATE | |
| CEFOXITIN SODIUM | MEPENZOLATE BROMIDE | OXCARBAZEPINE |
| SURAMIN | MERCAPTOPURINE | MELOXICAM SODIUM |
| CEFUROXIME SODIUM | METHENAMINE | CARVEDILOL |
| VIGABATRIN | METHICILLIN SODIUM | IRBESARTAN |
| LOMEFLOXACIN | METHOXSALEN | LEVOFLOXACIN |
| HYDROCHLORIDE | | |
| CEFAMANDOLE SODIUM | METHYLERGONOVINE | LITHIUM CITRATE |
| | MALEATE | |
| CEFMETAZOLE SODIUM | METHYLTHIOURACIL | GATIFLOXACIN |
| CEFOPERAZONE SODIUM | MICONAZOLE NITRATE | MIGLITOL |
| OFLOXACIN | NEOMYCIN SULFATE | ORLISTAT |
| BEZAFIBRATE | NITROFURAZONE | MOXIFLOXACIN |
| | | HYDROCHLORIDE |
| CETIRIZINE HYDROCHLORIDE | NITROMIDE | PIOGLITAZONE |
| | | HYDROCHLORIDE |
| PHENYLETHYL ALCOHOL | NORETHINDRONE ACETATE | DONEPEZIL HYDROCHLORIDE |
| MECLOCYCLINE | OXIDOPAMINE | FLUVASTATIN |
| SULFOSALICYLATE | HYDROCHLORIDE | |
| RIBOFLAVIN | OXYQUINOLINE | PIZOTYLINE MALATE |
| | HEMISULFATE | |
| ACEBUTOLOL | PENICILLIN G POTASSIUM | EXEMESTANE |
| HYDROCHLORIDE | | |
| ASPARTAME | PENICILLIN V POTASSIUM | TILMICOSIN |
| VARDENAFIL | PHENOLPHTHALEIN | FLUNIXIN MEGLUMINE |
| HYDROCHLORIDE | | |
| FLUORESCEIN | PHENYLEPHRINE | CLORSULON |
| | HYDROCHLORIDE | |
| NIACINAMIDE | PHENYLPROPANOLAMINE | ESTROPIPATE |
| | HYDROCHLORIDE | |
| PROPRANOLOL | PHYSOSTIGMINE | CLAVULANATE LITHIUM |
| HYDROCHLORIDE (+/−) | SALICYLATE | |
| METHSCOPOLAMINE | PILOCARPINE NITRATE | ALCLOMETAZONE |
| BROMIDE | | DIPROPIONATE |
| EDROPHONIUM CHLORIDE | PINDOLOL | ALENDRONATE SODIUM |
| THIOPENTAL SODIUM | PIPERACILLIN SODIUM | ACARBOSE |
| PENTOBARBITAL | PIPERAZINE | ROPINIROLE |
| PHENFORMIN | PIROXICAM | QUETIAPINE |
| HYDROCHLORIDE | | |
| PENFLURIDOL | POLYMYXIN B SULFATE | RIZATRIPTAN BENZOATE |
| PHTHALYSULFATHIAZOLE | PRAZIQUANTEL | FAMCICLOVIR |
| VINCRISTINE SULFATE | PRAZOSIN HYDROCHLORIDE | AMLODIPINE BESYLATE |
| OMEPRAZOLE | PREDNISOLONE | EZETIMIBE |
| ZOLMITRIPTAN | PREDNISOLONE ACETATE | OLMESARTAN MEDOXOMIL |
| DEBRISOQUIN SULFATE | PREDNISONE | CEFTIBUTEN |
| SULFADOXINE | PRIMAQUINE DIPHOSPHATE | CEFDINIR |
| FINASTERIDE | PRIMIDONE | SIBUTRAMINE |
| | | HYDROCHLORIDE |
| PENTETIC ACID | PROBENECID | PERINDOPRIL ERBUMINE |
| PROSCILLARIDIN | PROCAINAMIDE | ROSUVASTATIN CALCIUM |
| | HYDROCHLORIDE | |
| JOSAMYCIN | PROCAINE HYDROCHLORIDE | RAMIPRIL |
| REPAGLINIDE | PROCHLORPERAZINE | ESCITALOPRAM OXALATE |
| | EDISYLATE | |
| CROTAMITON | PROCYCLIDINE | DERACOXIB |
| | HYDROCHLORIDE | |
| CEFPROZIL | PROMAZINE | CILOSTAZOL |
| | HYDROCHLORIDE | |
| METHYLDOPATE | PROPANTHELINE BROMIDE | CITICOLINE |
| HYDROCHLORIDE | | |
| SULFAQUINOXALINE SODIUM | DEXPROPRANOLOL | APRAMYCIN |
| | HYDROCHLORIDE | |
| POTASSIUM p- | PROPYLTHIOURACIL | SERTRALINE |
| AMINOBENZOATE | | HYDROCHLORIDE |
| BETAMETHASONE VALERATE | PSEUDOEPHEDRINE | ALFLUZOSIN |
| | HYDROCHLORIDE | |
| ERYTHROMYCIN | PYRANTEL PAMOATE | TELITHROMYCIN |
| PROMETHAZINE | PYRAZINAMIDE | OXAPROZIN |
| HYDROCHLORIDE | | |
| SCOPOLAMINE | PYRILAMINE MALEATE | OXFENDAZOLE |
| HYDROBROMIDE | | |
| THEOPHYLLINE | PYRIMETHAMINE | AMITRAZ |
| TOLNAFTATE | PYRVINIUM PAMOATE | PEFLOXACINE MESYLATE |
| TRIMETHOBENZAMIDE | QUINACRINE | CHLOROPHYLLIDE Cu |
| HYDROCHLORIDE | HYDROCHLORIDE | COMPLEX Na SALT |
| VINBLASTINE SULFATE | QUINIDINE GLUCONATE | BIFONAZOLE |
| CLEBOPRIDE MALEATE | QUININE SULFATE | TYLOSIN TARTRATE |
| PIRACETAM | RACEPHEDRINE | SARAFLOXACIN |
| | HYDROCHLORIDE | HYDROCHLORIDE |
| GLUCONOLACTONE | RESERPINE | CLOPIDOL |
| AZLOCILLIN SODIUM | RESORCINOL | CHLORMADINONE ACETATE |
| CHOLINE CHLORIDE | RIFAMPIN | OXICONAZOLE NITRATE |
| ATORVASTATIN CALCIUM | ROXARSONE | AZAPERONE |
| OXYPHENCYCLIMINE | SALICYL ALCOHOL | TRANILAST |
| HYDROCHLORIDE | | |
| PROPAFENONE | SALICYLAMIDE | AZELASTINE |
| HYDROCHLORIDE | | HYDROCHLORIDE |
| FLUCONAZOLE | SODIUM SALICYLATE | KETANSERIN TARTRATE |
| LOVASTATIN | SISOMICIN SULFATE | FIPRONIL |
| ATROPINE OXIDE | SPECTINOMYCIN | DECOQUINATE |
| | HYDROCHLORIDE | |
| SENNOSIDE A | SPIRONOLACTONE | CEFDITORIN PIVOXIL |
| TENIPOSIDE | STREPTOMYCIN SULFATE | VALACYCLOVIR |
| | | HYDROCHLORIDE |
| TANNIC ACID | STREPTOZOSIN | DULOXETINE |
| | | HYDROCHLORIDE |
| CARPROFEN | SULFABENZAMIDE | NISOLDIPINE |
| HYDROXYCHLOROQUINE | SULFACETAMIDE | MONTELUKAST SODIUM |
| SULFATE | | |
| DIRITHROMYCIN | SULFADIAZINE | BENURESTAT |
| MEPIVACAINE | SULFAMERAZINE | BENZOXIQUINE |
| HYDROCHLORIDE | | |
| NILUTAMIDE | SULFAMETHAZINE | BISMUTH SUBSALICYLATE |
| AMINOLEVULINIC ACID | SULFAMETHIZOLE | BENZOYLPAS |
| HYDROCHLORIDE | | |
| PARAMETHADIONE | SULFAMETHOXAZOLE | BROMINDIONE |
| METAXALONE | SULFAPYRIDINE | CAPOBENIC ACID |
| CHLOROGUANIDE | SULFASALAZINE | ACETOHEXAMIDE |
| HYDROCHLORIDE | | |
| CLARITHROMYCIN | SULFATHIAZOLE | ETHOXZOLAMIDE |
| HYDROQUINONE | SULFINPYRAZONE | FLUCYTOSINE |
| NATEGLINIDE | SULFISOXAZOLE | FOMEPIZOLE |
| | | HYDROCHLORIDE |
| CANDESARTAN CILEXTIL | SULINDAC | GLIPIZIDE |
| ROSIGLITAZONE | TAMOXIFEN CITRATE | GUANFACINE |
| LOSARTAN | TERBUTALINE HEMISULFATE | D-LACTITOL MONOHYDRATE |
| HOMOSALATE | TETRACAINE | LEVOCARNITINE |
| | HYDROCHLORIDE | |
| SALICYLANILIDE | TETRACYCLINE | LOBENDAZOLE |
| | HYDROCHLORIDE | |
| PROPOFOL | TETRAHYDROZOLINE | METHYLENE BLUE |
| | HYDROCHLORIDE | |
| GRISEOFULVIN | THIABENDAZOLE | METHYLATROPINE NITRATE |
| BENAZEPRIL | THIMEROSAL | NITHIAMIDE |
| HYDROCHLORIDE | | |
| VALSARTAN | THIOGUANINE | PRALIDOXIME CHLORIDE |
| SALSALATE | THIORIDAZINE | PREDNISOLONE |
| | HYDROCHLORIDE | HEMISUCCINATE |
| HYDROCORTISONE | THIOTHIXENE | PYRIDOXINE |
| RIFAXIMIN | TIMOLOL MALEATE | RIMANTADINE |
| | | HYDROCHLORIDE |
| CANRENONE | TOBRAMYCIN | SULFISOXAZOLE ACETYL |
| MODAFINIL | TOLAZOLINE | TAURINE |
| | HYDROCHLORIDE | |
| CLIOQUINOL | TOLBUTAMIDE | THIAMINE |
| RANOLAZINE | TRANYLCYPROMINE | TRICLOSAN |
| | SULFATE | |
| DANTHRON | TRIACETIN | TRIMETHADIONE |
| ACEDAPSONE | TRIAMCINOLONE | ZINC UNDECYLENATE |
| ATOMOXETINE | TRIAMCINOLONE | UNDECYLENIC ACID |
| HYDROCHLORIDE | ACETONIDE | |
| DESOXYCORTICOSTERONE | TRIAMCINOLONE | CLINDAMYCIN PALMITATE |
| ACETATE | DIACETATE | HYDROCHLORIDE |
| TRAMADOL HYDROCHLORIDE | TRIAMTERENE | CEFONICID SODIUM |
| TERBINAFINE | TRICHLORMETHIAZIDE | IFOSFAMIDE |
| HYDROCHLORIDE | | |
| TOPIRAMATE | TRIFLUOPERAZINE | NETILMICIN SULFATE |
| | HYDROCHLORIDE | |
| GEMIFLOXACIN MESYLATE | TRIHEXYPHENIDYL | DOXORUBICIN |
| | HYDROCHLORIDE | |
| PRAVASTATIN SODIUM | TRIMEPRAZINE TARTRATE | METHYSERGIDE MALEATE |
| LEVALBUTEROL | TRIMETHOPRIM | SOLIFENACIN |
| HYDROCHLORIDE | | |
| METFORMIN | TRIOXSALEN | ACEPROMAZINE MALEATE |
| HYDROCHLORIDE | | |
| PREGABALIN | TRIPELENNAMINE CITRATE | BIPERIDEN |
| PHENOXYBENZAMINE | TRIPROLIDINE | DEXCHLORPHENIRAMINE |
| HYDROCHLORIDE | HYDROCHLORIDE | MALEATE |
| TOPOTECAN | TROPICAMIDE | DILOXANIDE FUROATE |
| HYDROCHLORIDE | | |
| PINACIDIL | TRYPTOPHAN | ETIDRONATE DISODIUM |
| VERAPAMIL | TUAMINOHEPTANE SULFATE | NATAMYCIN |
| HYDROCHLORIDE | | |
| PANTOPRAZOLE | TYROTHRICIN | NORGESTIMATE |
| LOPERAMIDE | UREA | TERAZOSIN |
| HYDROCHLORIDE | | HYDROCHLORIDE |
| PODOFILOX | URSODIOL | TIOCONAZOLE |
| LEVODOPA | VALPROATE SODIUM | ERGOTAMINE TARTRATE |
| RUTOSIDE (rutin) | VANCOMYCIN | ANAGRELIDE |
| | HYDROCHLORIDE | HYDROCHLORIDE |
| ZOMEPIRAC SODIUM | VIDARABINE | ETOMIDATE |
| SPARTEINE SULFATE | WARFARIN | LAMOTRIGINE |
| TESTOSTERONE PROPIONATE | XYLOMETAZOLINE | RALOXIFENE |
| | HYDROCHLORIDE | HYDROCHLORIDE |
| METHIMAZOLE | ACETARSOL | CEFPODOXIME PROXETIL |
| ENILCONAZOLE | MERBROMIN | TADALAFIL |
| FIROCOXIB | PHENACETIN | AMINOPENTAMIDE |
| LINDANE | PHENYLMERCURIC ACETATE | ARSANILIC ACID |
| ACRISORCIN | SULFANILAMIDE | PANTHENOL |
| PHENYL AMINOSALICYLATE | AZELAIC ACID | PHENTERMINE |
| TESTOSTERONE | PHENETHICILLIN POTASSIUM | TRIENTINE HYDROCHLORIDE |
| SANGUINARINE SULFATE | THEOBROMINE | TICLOPIDINE |
| | | HYDROCHLORIDE |
| alpha-TOCHOPHEROL | STRYCHNINE | TICARCILLIN DISODIUM |
| alpha-TOCHOPHERYL | ACONITINE | TETRAMIZOLE |
| ACETATE | | HYDROCHLORIDE |
| DACTINOMYCIN | YOHIMBINE | TOLTRAZURIL |
| | HYDROCHLORIDE | |
| MITOMYCIN C | ADENOSINE PHOSPHATE | TOREMIPHENE CITRATE |
| DICHLORVOS | KETOTIFEN FUMARATE | ROLIPRAM |
| TEMEFOS | BETAHISTINE | ROLITETRACYCLINE |
| | HYDROCHLORIDE | |
| MITOTANE | MOLSIDOMINE | PIPAMPERONE |
| IVERMECTIN | MYCOPHENOLIC ACID | PANCURONIUM BROMIDE |
| SODIUM NITROPRUSSIDE | OLEANDOMYCIN | FUMAZENIL |
| | PHOSPHATE | |
| SODIUM OXYBATE | OUABAIN | ALTRENOGEST |
| ETHYL PARABEN | ALBUTEROL (+/−) | BISOPROLOL FUMARATE |
| COUMARIN | ARECOLINE HYDROBROMIDE | FLUDARABINE PHOSPHATE |
| ACETAMINOPHEN | CAPTOPRIL | MUPIROCIN |
| ACETAZOLAMIDE | CIMETIDINE | TEICOPLANIN [A(2-1) shown] |
| ACETOHYDROXAMIC ACID | CLOZAPINE | EPIRUBICIN |
| | | HYDROCHLORIDE |
| ACETYLCHOLINE | HYDRASTINE (1R, 9S) | VECURONIUM BROMIDE |
| ACETYLCYSTEINE | LIDOCAINE | ALISKIREN HEMIFUMARATE |
| | HYDROCHLORIDE | |
| ADENOSINE | PHENTOLAMINE | ACAMPROSATE CALCIUM |
| | HYDROCHLORIDE | |
| ALLOPURINOL | BUTAMBEN | PREDNISOLONE SODIUM |
| | | PHOSPHATE |
| ALVERINE CITRATE | CEFACLOR | PREGNENOLONE SUCCINATE |
| AMANTADINE | IODIPAMIDE | DARIFENACIN |
| HYDROCHLORIDE | | HYDROBROMIDE |
| AMIKACIN SULFATE | LIOTHYRONINE | DESOXYMETASONE |
| AMILORIDE HYDROCHLORIDE | ALLANTOIN | BETAMETHASONE ACETATE |
| AMINOCAPROIC ACID | ALTHIAZIDE | ERYTHROSINE SODIUM |
| AMINOGLUTETHIMIDE | ADENINE | ISOFLUPREDNONE ACETATE |
| AMINOSALICYLATE SODIUM | AMINACRINE | BETAMETHAZONE SODIUM |
| | | PHOSPHATE |
| AMITRIPTYLINE | BEKANAMYCIN SULFATE | MELENGESTROL ACETATE |
| HYDROCHLORIDE | | |
| AMODIAQUINE | BUDESONIDE | PHTHALYLSULFACETAMIDE |
| DIHYDROCHLORIDE | | |
| AMOXICILLIN | BRUCINE | TRICHLORFON |
| AMPHOTERICIN B | CANRENOIC ACID, | BEPHENIUM |
| | POTASSIUM SALT | HYDROXYNAPTHOATE |
| AMPICILLIN SODIUM | CHENODIOL | DIPERODON |
| | | HYDROCHLORIDE |
| AMPROLIUM | CHOLECALCIFEROL | DIATRIZOIC ACID |
| ANTAZOLINE PHOSPHATE | CINCHONIDINE | PANTOTHENIC ACID(d) Na salt |
| ANTHRALIN | CINCHONINE | DESONIDE |
| ANTIPYRINE | COENZYME B12 | GLYCOPYRROLATE |
| APOMORPHINE | CHOLESTEROL | ITRACONAZOLE |
| HYDROCHLORIDE | | |
| ASPIRIN | PIPERINE | OCTISALATE |
| ATROPINE SULFATE | ETOPOSIDE | RIBOFLAVIN 5-PHOSPHATE |
| | | SODIUM |
| AUROTHIOGLUCOSE | DEHYDROCHOLIC ACID | SELEGILINE |
| | | HYDROCHLORIDE |
| AZATHIOPRINE | FLUMEQUINE | CEFTAZIDIME |
| BACITRACIN | FLUNARIZINE | GABAPENTIN |
| | HYDROCHLORIDE | |
| BACLOFEN | FLUPHENAZINE | ELETRIPTAN |
| | HYDROCHLORIDE | HYDROBROMIDE |
| BECLOMETHASONE | FLUTAMIDE | ARIPIPRAZOLE |
| DIPROPIONATE | | |
| BENSERAZIDE | DROPERIDOL | ZILEUTON |
| HYDROCHLORIDE | | |
| BENZETHONIUM CHLORIDE | FAMOTIDINE | METHYLPHENIDATE |
| | | HYDROCHLORIDE |
| BENZOCAINE | ETODOLAC | RABEPRAZOLE SODIUM |
| BENZTHIAZIDE | FENOTEROL | RISEDRONATE SODIUM |
| | HYDROBROMIDE | HYDRATE |
| beta-CAROTENE | FENBUFEN | SUCRALOSE |
| BETAMETHASONE | MEBEVERINE | COLISTIN SULFATE |
| | HYDROCHLORIDE | |
| BETHANECHOL CHLORIDE | ACECLIDINE | ARSENIC TRIOXIDE |
| BISACODYL | CAPSAICIN | CLONAZEPAM |
| BITHIONATE SODIUM | FAMPRIDINE | BENZBROMARONE |
| BROMOCRIPTINE MESYLATE | NICERGOLINE | BROMPERIDOL |
| BUSULFAN | SPIPERONE | CYPROHEPTADINE |
| | | HYDROCHLORIDE |
| CAFFEINE | ERYTHROMYCIN ESTOLATE | CLOFAZIMINE |
| CAMPHOR (1R) | ESTRADIOL PROPIONATE | BENZYDAMINE |
| | | HYDROCHLORIDE |
| CAPREOMYCIN SULFATE | ESTRADIOL BENZOATE | DOXAZOSIN MESYLATE |
| CARBACHOL | RETINOL | ISOETHARINE MESYLATE |
| CARBAMAZEPINE | ISOTRETINON | FLORFENICOL |
| CARBENICILLIN DISODIUM | MESNA | ETHYNODIOL DIACETATE |
| CARBINOXAMINE MALEATE | TRETINON | ORNIDAZOLE |
| CARISOPRODOL | BRETYLIUM TOSYLATE | OXANTEL PAMOATE |
| CEFADROXIL | FOSCARNET SODIUM | PROTRYPTYLINE |
| | | HYDROCHLORIDE |
| CEFOTAXIME SODIUM | CEFSULODIN SODIUM | PHYTONADIONE |
| CEPHALOTHIN SODIUM | FOSFOMYCIN CALCIUM | DENATONIUM BENZOATE |
| CEPHAPIRIN SODIUM | CEFAMANDOLE NAFATE | MESALAMINE |
| CEPHRADINE | LIOTHYRONINE (L-isomer) | ETHAMIVAN |
| | SODIUM | |
| CETYLPYRIDINIUM CHLORIDE | ALRESTATIN | AZTREONAM |
| CHLORAMBUCIL | PROADIFEN | TYLOXAPOL |
| | HYDROCHLORIDE | |
| CHLORAMPHENICOL | CARBOPLATIN | THIAMYLAL SODIUM |
| PALMITATE | | |
| CHLORAMPHENICOL | CISPLATIN | CHLORDIAZEPOXIDE |
| HEMISUCCINATE | | |
| CHLORAMPHENICOL | ZIDOVUDINE [AZT] | ASTEMIZOLE |
| CHLORCYCLIZINE | AZACITIDINE | ACECAINIDE |
| HYDROCHLORIDE | | HYDROCHLORIDE |
| CHLORHEXIDINE | CYCLOHEXIMIDE | FLUROTHYL |
| CHLOROCRESOL | TINIDAZOLE | ALPRENOLOL |
| CHLOROQUINE DIPHOSPHATE | CARBIDOPA | AMIODARONE |
| | | HYDROCHLORIDE |
| CHLOROTHIAZIDE | ETHOSUXIMIDE | BUSPIRONE |
| | | HYDROCHLORIDE |
| CHLOROTRIANISENE | PIPERIDOLATE | LOXAPINE SUCCINATE |
| | HYDROCHLORIDE | |
| CHLOROXYLENOL | ANISINDIONE | DIAZOXIDE |
| CHLORPHENIRAMINE (S) | CYCLOSPORINE | DILTIAZEM HYDROCHLORIDE |
| MALEATE | | |
| CHLORPROMAZINE | ASCORBIC ACID | GLYBURIDE |
| CHLORPROPAMIDE | MENADIONE | MIANSERIN |
| | | HYDROCHLORIDE |
| CHLORTETRACYCLINE | SALICIN | VESAMICOL |
| HYDROCHLORIDE | | HYDROCHLORIDE |
| CHLORTHALIDONE | MONENSIN SODIUM (monensin | NIZATIDINE |
| | A is shown) | |
| CHLORZOXAZONE | ABAMECTIN | PENTYLENETETRAZOL |
| CICLOPIROX OLAMINE | BENZOIC ACID | NICOTINE DITARTRATE |
| CINOXACIN | BENZYL BENZOATE | TACRINE HYDROCHLORIDE |
| CLEMASTINE | BENZOYL PEROXIDE | DIMERCAPROL |
| CLIDINIUM BROMIDE | BETAINE HYDROCHLORIDE | METOLAZONE |
| CLINDAMYCIN | BIOTIN | AMOXAPINE |
| HYDROCHLORIDE | | |
| CLOMIPHENE CITRATE | AKLOMIDE | BUTYL PARABEN |
| CLONIDINE HYDROCHLORIDE | NICOTINYL ALCOHOL | DECAMETHONIUM BROMIDE |
| | TARTRATE | |
| CLOTRIMAZOLE | FLOXURIDINE | CARBADOX |
| CLOXACILLIN SODIUM | ALTRETAMINE | ENROFLOXACIN |
| CLOXYQUIN | AMINOHIPPURIC ACID | DEXPANTHENOL |
| COLCHICINE | MEFLOQUINE | NONOXYNOL-9 |
| COLISTIMETHATE SODIUM | ADIPHENINE | DOCOSANOL |
| | HYDROCHLORIDE | |
| CORTISONE ACETATE | QUINAPRIL | OCTODRINE |
| | HYDROCHLORIDE | |
| COTININE | AMIFOSTINE | ANIRACETAM |
| CRESOL | AMIPRILOSE | PENTOXIFYLLINE |
| CROMOLYN SODIUM | TIAPRIDE HYDROCHLORIDE | AZTREONAM |
| CYCLIZINE | BACAMPICILLIN | CEFAZOLIN SODIUM |
| | HYDROCHLORIDE | |
| CYCLOPENTOLATE | CYPROTERONE ACETATE | TUBOCURARINE CHLORIDE |
| HYDROCHLORIDE | | |
| CYCLOPHOSPHAMIDE | CYTARABINE | TOLMETIN SODIUM |
| HYDRATE | | |
| CYCLOSERINE | DACARBAZINE | BENDROFLUMETHIAZIDE |

TABLE 6
Top 200 Brand Name Drugs in 2008

| | |
|---|---|
| 1 | Lipitor |
| 2 | Nexium |
| 3 | Plavix |
| 4 | Advair Diskus |
| 5 | Prevacid |
| 6 | Seroquel |
| 7 | Singulair |
| 8 | Effexor XR |
| 9 | OxyContin |
| 10 | Actos |
| 11 | Lexapro |
| 12 | Abilify |
| 13 | Topamax |
| 14 | Cymbalta |
| 15 | Zyprexa |
| 16 | Valtrex |
| 17 | Crestor |
| 18 | Vytorin |
| 19 | Lamictal |
| 20 | Celebrex |
| 21 | Lantus |
| 22 | Levaquin |
| 23 | Adderall XR |
| 24 | Lyrica |
| 25 | Diovan |
| 26 | Tricor |
| 27 | Flomax |
| 28 | Risperdal |
| 29 | Diovan HCT |
| 30 | Zetia |
| 31 | Aricept |
| 32 | Spiriva |
| 33 | Concerta |
| 34 | Aciphex |
| 35 | Imitrex Oral |
| 36 | Lidoderm |
| 37 | Keppra |
| 38 | Viagra |
| 39 | Atripla |
| 40 | Lovenox |
| 41 | Januvia |
| 42 | Nasonex |
| 43 | Ambien CR |
| 44 | Provigil |
| 45 | Geodon Oral |
| 46 | Truvada |
| 47 | Lunesta |
| 48 | Enbrel |
| 49 | Actonel |
| 50 | CellCept |
| 51 | Humalog |
| 52 | Detrol LA |
| 53 | Depakote ER |
| 54 | Cozaar |
| 55 | Pulmicort Respules |
| 56 | Niaspan |
| 57 | Wellbutrin XL |
| 58 | Chantix |
| 59 | Budeprion XL |
| 60 | Byetta |
| 61 | Yaz |
| 62 | Prograf |
| 63 | Namenda |
| 64 | Arimidex |
| 65 | Combivent |
| 66 | Cialis |
| 67 | Flovent HFA |
| 68 | Protonix |
| 69 | Premarin Tabs |
| 70 | Suboxone Hyzaar |
| 71 | Hyzaar |
| 72 | ProAir HFA |
| 73 | Reyataz |
| 74 | Benicar HCT |
| 75 | Synthroid |
| 76 | Avandia |
| 77 | Boniva |
| 78 | Strattera |
| 79 | Polymagma Plain |
| 80 | Skelaxin |
| 81 | Evista |
| 82 | Asacol |
| 83 | Depakote |
| 84 | Xalatan |
| 85 | Humira |
| 86 | Benicar |
| 87 | Gleevec |
| 88 | AndroGel |
| 89 | Enbrel Sureclick |
| 90 | Avelox |
| 91 | Fantanyl Oral Citra |
| 92 | Lovaz |
| 93 | RenaGel |
| 94 | Avapro |
| 95 | Humira Pen |
| 96 | Vyvanse |
| 97 | Kaletra |
| 98 | Xopenex |
| 99 | Copaxone |
| 100 | Avodart |
| 101 | Femara |
| 102 | Avalide |
| 103 | Ortho TriCyclen |
| | Lo |
| 104 | Sensipar |
| 105 | Aldara |
| 106 | NovoLog Mix |
| 107 | Restasis |
| 108 | Mirapex |
| 109 | Yasmin 28 |
| 110 | Solodyn |
| 111 | Lantus SoloSTAR |
| 112 | Norvir |
| 113 | Focalin XR |
| 114 | Actoplus Met |
| 115 | Vesicare |
| 116 | Forteo |
| 117 | Allegra-D |
| 118 | Procrit. |
| 119 | Nasacort AQ |
| 120 | Tarceva |
| 121 | Combivir |
| 122 | Tamiflu |
| 123 | Avonex |
| 124 | NuvaRing |
| 125 | Coreg CR |
| 126 | Epzicom |
| 127 | Levemir |
| 128 | Duragesic |
| 129 | Risperdal |
| | Consta |
| 130 | Zyvox |
| 131 | Tussionex |
| 132 | Invega |
| 133 | Fosamax |
| 134 | Kadian |
| 135 | Levitra |
| 136 | Differin |
| 137 | Astelin |
| 138 | Lumigan |
| 139 | Symbicort |
| 140 | Janumet |
| 141 | Xeloda |
| 142 | Clarinex |
| 143 | Proventil |
| | HFA |
| 144 | Humalog Mix |
| | 75/25 Pn |
| 145 | BenzaClin |
| 146 | Vigamox |
| 147 | Foxamax Plus D |
| 148 | Maxalt |
| 149 | Cosopt |
| 150 | Requip |
| 151 | Relpax\ |
| 152 | Patanol |
| 153 | Casodex |
| 154 | Welchol |
| 155 | Ciprodex Otic |
| 156 | Viread |
| 157 | Catapres-TTS |
| 158 | Loestrin 24 Fe |
| 159 | Thalomid |
| 160 | Alphagan P |
| 161 | Endocet |
| 162 | Revlimid |
| 163 | Avandamet |
| 164 | Maxalt MLT |
| 165 | Altace |
| 166 | Budeprion SR |
| 167 | Pegasys |
| 168 | Ultram ER |
| 169 | Fentora |
| 170 | Asmanex |
| 171 | Rhinocort Aqua |
| 172 | Temodar |
| 173 | Micardis HCT |
| 174 | Sotret |
| 175 | Trizivir |
| 176 | Enablex |
| 177 | Isentress |
| 178 | TobraDex |
| 179 | Trileptal |
| 180 | Sustiva |
| 181 | Amitiza |
| 182 | Micardis |
| 183 | Zovirax |
| | Topical |
| 184 | Ocella |
| 185 | Propecia |
| 186 | Taclonex |
| 187 | Actiq |
| 188 | Valcyte |
| 189 | Klor-Con |
| 190 | Atacand |
| 191 | Doryx |
| 192 | Veramyst |
| 193 | Avinza |
| 194 | Allegra-D 24 |
| | Hour |
| 195 | Opana ER |
| 196 | Zomig |
| 197 | Humulin 70/30 |
| 198 | Prempro |
| 199 | Humulin N |
| 200 | Xopenex HFA |

1. A method for creating a chemical compound, namely A-B, from two chemical fragments, namely A and B, wherein the chemical compound binds to a target protein, the method comprising:
(a) methylating one of the chemical fragments, A, at one or more nucleophilic atoms to obtain a ¹³CH₃-methylated analog of A, namely A-¹³CH₃, by performing an alkylation reaction;
(b) forming a mixture comprising: (1) A-¹³CH₃; (2) the other chemical fragment, B, which comprises an allylic or benzylic methyl group, and (3) the target protein;
(c) determining whether both A-¹³CH₃ and B bind to the target protein in the mixture such that the methyl group of A-¹³CH₃ and the methyl group of B are located no more than 5 angstroms apart; and if so
(d) performing the alkylation reaction of step (a) using A and B as reagents in order to covalently attach A and B via the methyl group carbon atom of B to obtain the chemical compound A-B, optionally wherein the methyl group B first is halogenated and reacts with the nucleophilic atom of A.

2. The method of claim 1, wherein step (c) comprises performing a nuclear magnetic resonance experiment on the mixture and determining whether a Nuclear Overhauser Effect (NOE) is occurring.

3. The method of claim 2, wherein determining whether an NOE is occurring comprises performing a ¹³C-filtered measurement either in a single dimension or in two dimensions.

4. The method of claim 2, wherein the mixture further comprises a biological sample that comprises the target protein.

5. The method of claim 4, further comprising performing nuclear magnetic resonance on a mixture formed from: (1) A-¹³CH₃; (2) the other chemical fragment, B, which comprises an allylic or benzylic methyl group, and (3) the biological sample after the target protein has been removed from the biological sample.

6. The method of claim 4, wherein the biological sample comprises an extract of brain tissue, heart tissue, or liver tissue, which optionally first has been purified on an affinity column that comprises a ligand for the target protein.

7. The method of claim 1, wherein the target protein is a KCNQ (Kv7) channel protein.

8. The method of claim 1, wherein the chemical fragment A comprises a nucleophilic atom selected from a nucleophilic carbon, a nucleophilic oxygen, or a nucleophilic sulfur atom and the chemical fragment A is methylated at the nucleophilic atom in step (a) and the chemical fragment A is covalently attached to chemical fragment B via forming a bond between the nucleophilic atom of chemical fragment A and the methyl group carbon atom of chemical fragment B in step (d) after the methyl group carbon atom of chemical fragment B has been halogenated.

9. The method of claim 1, wherein the chemical fragment A has a formula selected from:

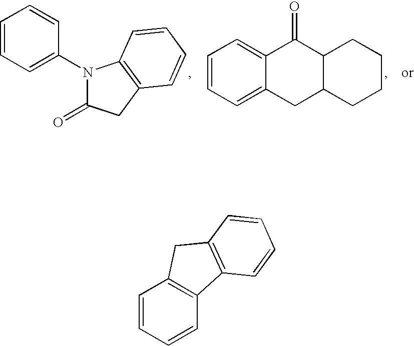

10. The method of claim 1, wherein the di-methylated chemical fragment A has a formula selected from:

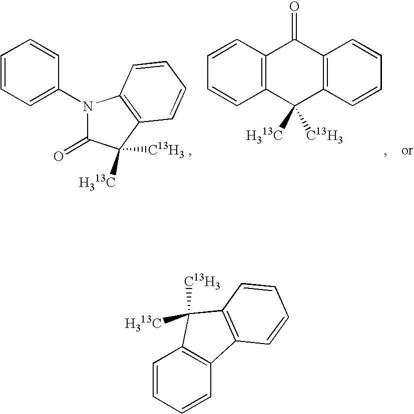

11. The method of claim 1, wherein the chemical fragment A is a compound selected from the list of compounds in Table 1.

12. The method of claim 1, wherein the chemical fragment A is obtained by halogenating a compound in Table 2 or Table 3 at an allylic or benzylic methyl group and subsequently reacting the halogenated compound with a thiol anion or an oxy anion.

13. The method of claim 1, wherein the chemical fragment B is a compound selected from the list of compounds in Table 2 or Table 3.

14. The method of claim 1, wherein the chemical fragment B includes a fused ring moiety selected from a quinoline, an isoquinoline, and an acridine.

15. The method of claim 1, wherein the chemical fragment B has a formula selected from:

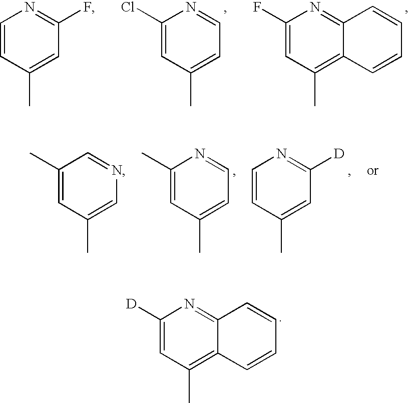

16. The method of claim 1, wherein the alkylation reaction comprises:
(i) reacting the chemical fragment A with a strong base and deprotonating the chemical fragment A at a nucleophilic atom selected from carbon, oxygen, or sulfur; and
(ii) reacting the deprotonated chemical fragment A with a methyl halide thereby methylating the chemical fragment A at the nucleophilic atom.

17. The method of claim 1, wherein the alkylation reaction of step (d) comprises:
(i) reacting the chemical fragment A with a strong base and deprotonating the chemical fragment A at a nucleophilic atom selected from carbon, oxygen, or sulfur;
(ii) halogenating the methyl group of the chemical fragment B to obtain a derivative of chemical fragment B having a halogenated methyl group; and
(iii) reacting the deprotonated chemical fragment A with the derivative of chemical fragment B having the halogenated methyl group, thereby forming a C—C, C—O, or C—S bond between the deprotonated atom of the chemical fragment A and the methyl group carbon of the chemical fragment B.

18. The method of claim 17, wherein halogenating is performed by reacting the chemical fragment B with N-bromosuccinimide (NBS) or N-chlorosuccinimide (NCS).

19. A method for creating a chemical compound, namely A-B, from two chemical fragments, namely A and B, wherein the chemical compound binds to a KCNQ (Kv7) channel protein, the method comprising:
(a) methylating one of the chemical fragments, A, at one or more positions to obtain a ¹³CH₃-methylated analog of A, namely A-¹³CH₃, by performing an alkylation reaction, wherein a di-methylated form of A, namely has a formula selected from: 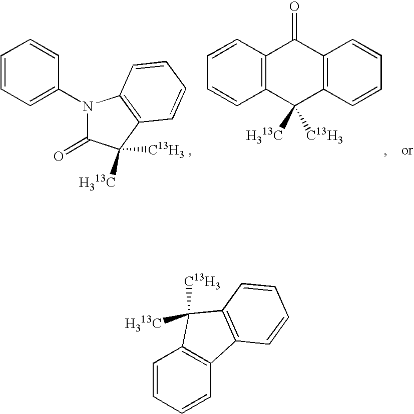
(b) forming a mixture comprising: (1) the di-methylated form of A; (2) the other chemical fragment, B, which is selected from compounds listed in Table 2 or Table 3, and (3) the KCNQ (Kv7) channel protein;
(c) determining whether both A-¹³CH₃ and B bind to the target protein in the mixture such that the methyl group of A-¹³CH₃ and the methyl group of B are located no more than 5 angstroms apart; and if so
(d) performing the alkylation reaction of step (a) using A and B as reagents in order to covalently attached A and B via the methyl group carbon atom of B to obtain the chemical compound A-B.

20. The method of claim 19, wherein B is a methyl-substituted pyridine compound.
